SEQUENCE LISTING (3 sequences)

SEQ ID NO: 1 - protein, 866 aa, Artificial
  R2 strain envelope protein (gp 160) of HIV type 1
MRVKGIRRNYQHWWGWGTMLLGLLMICSATEKLWVTVYYGVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSNSEGTIKGGEMKNCSFNIATSIGDKMQKEYALLYKLDIEPIDNDNTSYRLISCNTSVITQACPKISFEPIPIHYCAPAGFAILKCNDKKFSGKGSCKNVSTVQCTHGIRPVVSTQLLLNGSLAEEEVVIRSENFTNNAKTIIVQLREPVKINCSRPNNNTRKSIPMGPGRAFYTTGQIIGDIRQAHCNISKTNWTNALKQVVEKLGEQFNKTKIVFTNSSGGDPEIVTHSFNCAGEFFYCNTTQLFDSIWNSENGTWNITRGLNNTGRNDTITLPCRIKQIINRWQEVGKAMYAPPIKGNISCSSNITGLLLTRDGGKDDNSRDGNETFRPGGGDMRDNWRSELYKYKVVKIEPLGVAPTKAKRRVVQREERAVGLGAMFFGFLGAAGSTMGAASVTLTVQARQLLSGIVQQQSNLLRAIEAQQHLLQLTVWGIKQLQARILAVERYLKDQQLLGIWGCSGKLICTTTVPWNASWSKNKTLEAIWNNMTWMQWDKEIDNYTSLIYSLIEESPIQQEKNEQELLELDKWANLWNWFDISNWLWYIKIFIMIVGGLVGLRIVFVVLSIVNRVRQGYSPLSFQTRLPAPRGPDRPEEIEEEGGDRDRDRSGLLVDGFLTLIWVDLRSLCLFSYHRLRDLLLIVTRIVELLGRRGWEILKYWWNLLQYWSQELKNSAVSLFNATAIAVAEGTDRVIQVLQRVGRALLHIPTRIRQGLERALL

SEQ ID NO: 2 - protein, 13 aa, Artificial
  Amino acids 313 to 325 of HIV R2 strain envelope protein (gp 160)
PMGPGRAFYTTGQ

SEQ ID NO: 3 - protein, 8 aa, Artificial
  Amino acids 164 to 171 of HIV R2 strain envelope protein (gp 160)
FNIATSIG

(19) United States
(12) Patent Application Publication
(10) Pub. No.: US 2011/0293697 A1
(43) Pub. Date: Dec. 1, 2011

(54) HIV-1 Immunogenic Compositions

(76) Inventors: Gerald V. Quinnan, Rockville, MD (US); Christopher C. Broder, Silver Spring, MD (US); Gerald H. Voss, Grez-Doiceau (BE); Nathalie L. Mathy, Ramillies (BE)

(21) Appl. No.: 13/081,756

(22) Filed: Apr. 7, 2011

Related U.S. Application Data
(63) Continuation of application No. 12/852,121, filed on Aug. 6, 2010, now abandoned.
Application No. 12/852,121 is a continuation of application No. 12/299,075, now abandoned.
(60) Provisional application No. 60/798,718, filed on May 9, 2006.

(51) Int. Cl. A61K 9/127; A61P 37/04; A61P 31/18; A61K 39/21

(52) U.S. Cl. 424/450; 424/188.1

(57) ABSTRACT

The present invention encompasses vaccine and/or immunogenic compositions against HIV and their methods of use for the prevention and/or treatment of HIV infection and/or AIDS. The vaccine and/or immunogenic compositions may contain an isolated HIV protein or fragment thereof, an adjuvant comprising a Toll like receptor (TLR) 4 ligand, in combination with a saponin.

### CROSS-REFERENCE TO RELATED APPLICATIONS

This application claims the benefit of U.S. Provisional Application 60/798,718 (filed May 9, 2006) which is hereby incorporated by reference in its entirety.

### ACKNOWLEDGMENT OF FEDERAL SUPPORT

The present invention arose in part from research funded by NIH grants AI37438 and AI064070.

### FIELD OF THE INVENTION

The present invention relates to the prevention and treatment of HIV infection and/or AIDS.

### BACKGROUND OF THE INVENTION

Human immunodeficiency virus type-1 (HIV-1) is the etiologic agent of acquired immunodeficiency syndrome (AIDS). The HIV-1 strains that account for the global pandemic are designated the group M (major) strains, which are classified into some ten genetic subtypes or clades. The HIV-1 M group subtypes are phylogenetically associated groups of HIV-1 sequences, and are labeled A, B, C, D, F1, F2, G, H, J and K, as well as sixteen circulating recombinant forms (Korber et al. (1999) Human Retroviruses and AIDS (vol. III) 492-505). The sequences within any one subtype are more similar to each other than to sequences from other subtypes throughout their genomes. These subtypes represent different lineages of HIV, and have some geographical associations. Former subtypes E and I are both now defined as circulating recombinant forms (CRF) (Korber et al. (1999) Human Retroviruses and AIDS (vol. III) 492-505). Untreated HIV-1 infection is generally characterized by a progressive and irreversible decline in the number of CD4+ lymphocytes (Pantaleo et al. (1993) N. Eng. J. Med. 328, 327-335) and an increase in the viral burden (Pantaleo et al. (1993) Nature 362, 355-358; Piatak et al. (1993) Lancet 341, 1099).

The development of a successful vaccine against HIV infection or a vaccine agent capable of preventing HIV disease progression has been a public health goal for over 15 years. One of the immune responses that may be required to elicit a protective immune response against HIV infection is the generation of antibodies that are virus neutralizing.

Previous subunit HIV-1 envelope vaccine efforts using monomeric forms of gp120 or gp160 have been shown to be immunogenic in small animals, primates and humans but the antibody responses, though capable of neutralizing TCLA HIV-1 isolates, have had limited neutralizing activity against primary HIV-1 isolates (Belshe et al. (1994) JAMA 272, 475-480; Hanson (1994) AIDS Res. Hum. Retrovir. 10, 645-648; Kahn et al. (1994) J. Infect. Dis. 170, 1288-1291; Mascola et al. (1996) J. Infect. Dis. 173, 340-348; Matthews (1994) AIDS Res. Hum. Retrovir. 10, 631-632; Schwartz et al. (1993) Lancet 342, 69-73; Wrin et al. (1994) AIDS 8, 1622-1623). Furthermore, several individuals enrolled in clinical trials of candidate monomeric gp120 subunit vaccines became HIV-1 infected despite receiving the full vaccination regimen (Connor et al. (1998) J. Virol. 72, 1552-1576; Kahn et al. (1995) J. Infect. Dis. 171, 1343-1347; McElrath et al. (1996) Proc. Natl. Acad. Sci. USA 93, 3972-3977) and these infections were not correlated with infecting strain (Connor et al. (1998) J. Virol. 72, 1552-1576). These results may be attributable to the inability of these monomeric gp120 vaccines to elicit antibodies specific for conserved, discontinuous epitopes, since the majority of antibodies are focused primarily to linear epitopes poorly accessible on cell surface expressed gp120/gp41 (VanCott et al. (1995) J. Immunol. 155, 4100-4110). These data suggest that monomeric gp120 based upon TCLA isolates may lack important structural properties critical for the ability to induce broadly reactive and neutralizing antibody. These structural properties may be related to the choice of vaccine strain since TCLA and primary isolates have been demonstrated to have significant phenotypic differences with respect to co-receptor usage (Alkhatib et al. (1996) Science 272, 1955-1958; Deng et al. (1996) Nature 381, 661-666; Drajic et al. (1996) Nature 381, 667-673; Feng et al. (1996) Science 272, 872-877) and susceptibility to antibody or serum mediated neutralization (Ashkenazi et al. (1991) Proc. Natl. Acad. Sci. USA 88, 7056-7060; Brighty et al. (1991) Proc. Natl. Acad. Sci. USA 88, 7802-7805; Daar et al. (1990) Proc. Natl. Acad. Sci. USA 87, 6574-6578; Moore et al. (1995) J. Virol. 69, 101-109; Robb et al. (1992) J. Acquired Immune Defic. Syndr. 5, 1224-1229; Sawyer et al. (1994) J. Virol. 68, 1342-1349). However, monomeric gp120 from strains MN and SF2 have also been shown to protect chimpanzees against homologous primary isolate HIV-1_{SF2} challenge (Berman et al. (1996) J. Infect. Dis. 173, 52-59; Girard et al. (1995) J. Virol. 69, 6239-6248; el-Amad et al. (1995) AIDS 9, 1313-1322). Recently, chimpanzees primed with adenovirus expressing gp160 and boosting with rgp120_{SF2} elicited neutralizing antibody against primary isolates using CXCR4 co-receptor and non PITA-stimulated PBMC (Zolla-Pazner et al. (1998) J. Virol. 72, 1052-1059). The latter indicate the possibility of enhanced functional antibody properties when used in the context of a prime boost immunization regiment.

There are several potently neutralizing monoclonal antibodies which map to regions accessible on monomeric gp120 (Trkola et al. (1995) J. Virol. 69, 6609-6617; Trkola et al. (1996) J. Virol. 70, 1100-1108; Tilley et al. (1991) Res. Virol. 142, 247-259; Thali et al. (1992) J. Virol. 66, 5635-5641; Thali et al. (1991) J. Virol. 65, 6188-6193; Gorny et al. (1992) J. Virol. 66, 7538-7542; Gorny et al. (1993) J. Immunol. 150, 635-643; Gorny et al. (1994) J. Virol. 68, 8312-8320; Conley et al. (1994) Proc. Natl. Acad. Sci. USA 91, 3348-3352; Conley et al. (1994) J. Virol. 68, 6994-7000; Burton et al. (1994) Science 266, 1024-1027; Barbas et al. (1994) Proc. Natl. Acad. Sci. USA 91, 3809-3813; Moore et al. (1995) J. Virol. 69, 122-130; Posner et al. (1993) J. Acquired Immune Defic. Syndr. 6, 7-14; Muster et al. (1993) J. Virol. 67, 6642-6647) and it remains to be determined why these neutralizing epitopes, present on monomeric gp120, are not immunogenic when presented in the context of a vaccine. The majority of the broadly anti-gp120 neutralizing monoclonal antibodies are directed to conformational epitopes that have been particularly difficult to elicit using monomeric HIV-1 subunit vaccines. Studies designed to correlate antibody binding with functional capacity have shown that monomeric gp120 is not as predictive as oligomeric gp160 in predicting neutralization capacity (Moore et al. (1995) J. Virol. 69, 101-109; Moore et al. (1994) J. Virol. 68, 469-484; Sattentau et al. (1995) J. Exp. Med. 182, 185-196; Stamatatos et al. (1995) J. Virol. 69, 6191-6198; Sullivan et al. (1995) J. Virol. 69, 4413-4422; Fouts et al. (1997) J. Virol. 71, 2779-2785), most likely attributable to many epitopes on gp120 being hidden in the context of membrane expressed oligomeric gp120/gp41.

Explanations of the difficulty in inducing neutralizing antibodies to conserved, conformational epitopes may include structural instability of monomeric forms of gp120, which may perhaps be stabilized within the context of the proper quaternary structure of the HIV-1 envelope glycoprotein. The HIV-1 envelope glycoprotein gp160 is known to exist as a multimer (trimers or tetramers) on the surface of a virion (Earl et al. (1990) Proc. Natl. Acad. Sci. USA 87, 648-652; Pinter et al. (1989) J. Virol. 2674-2679; Schawaller et al. (1989) Virology 172, 367-369; Thomas et al. (1991) J. Virol. 65, 3797-3803). Recent structural data on gp41 showed peptides corresponding to two regions of gp41 with substantial alpha-helical content formed an alpha-helical coiled-coil trimer, resembling functionally the hemaglutinin fusion protein (Chan et al. (1997) Cell 89, 263-273; Weissenhorn et al. (1997) Nature 387, 426-430) consistent with previous biochemical data demonstrating that gp41 forms oligomers (trimers) with significant alpha-helical content in the absence of gp120 (Weissenhorn et al. (1996) EMBO J 15, 1507-1514). Another recent study demonstrated that gp41 derived from gp160 expression in mammalian cells forms tetramers indicating the possibility that regions outside of the alpha helical gp41 sequences may impact on overall quaternary structure of gp41 (McInerney et al. (1998) J. Virol. 72, 1523-1533). It has been shown that immunization of mice with oligomeric gp140 results in the induction of a number of mAbs with specificity to oligomeric-specific or sensitive epitopes within gp41 (Broder et al. (1994) Proc. Natl. Acad. Sci. USA 91, 11699-11703; Earl et al. (1994) J. Virol. 68, 3015-3026). Further mapping of these responses indicated six antigenic determinants of which 3 were conformational epitopes dependent upon oligomeric structure (Earl et al. (1997) J. Virol 71, 2674-2694). These mAbs were found to compete with HIV-1 serum and were cross reactive with HIV-1 gp41 from highly divergent isolates indicating these epitopes to be substantially conserved. However, HIV-1 neutralizing activity of these mAbs has not been determined and previous studies have demonstrated that many gp41 specific mAbs lack significant neutralizing activity perhaps due to the majority of epitopes being blocked by association with gp120 (Sattentau et al. (1995) Virology 206, 713-717).

Several studies have demonstrated that passively-transferred envelope-specific neutralizing antibody can protect against SHIV disease and/or infection in non-human primates (Parren et al. (2001) J. Virol. 75, 8340-8347; Mascola et al. (2000) Nature Medicine 6, 207-210; Mascola et al. (1999) J. Virol. 73, 4009-4018; Baba et al. (2000) Nature Medicine 6, 200-206; Shibata et al. (1999) Nature Medicine 5, 204-210) highlighting the potential critical role of HIV-specific neutralizing antibody in vaccine efficacy. The essential antibody functional property is neutralizing capacity against the challenge virus. Vaccine-induced broadly neutralizing antibodies (different than the antibodies elicited to HIV infection used in the passive transfer studies) have been difficult to achieve. Recent encouraging developments have shown the ability of DNA and recombinant viral vaccination strategies to induce viral-specific CD8 T cell responses (Amara et al. (2001) Science 292, 69-74; Barouch et al. (2001) J. Virol. 75, 5151-5158; Barouch et al. (2000) Science 290, 486-492). These responses, in the absence of measurable neutralizing antibody, have provided some level of protection (not sterilizing) from disease after pathogenic challenge. The current goal of inducing more potent neutralizing antibody and combining these with vaccination strategies inducing CD8 T cell responses may provide increased levels of protection. The goal remains to continue research into novel subunit envelope vaccines towards the induction of neutralizing antibody.

Previously, an isolated HIV-1 envelope sequence was identified which, when administered to rabbits, resulted in the production of antibodies with a broadly cross-reactive response against multiple strains of HIV-1 in vitro (WO 00/07631). The present invention advances these earlier findings thru identification of an adjuvant system which can be used in combination with these envelope proteins to provide improved and unexpected findings of an enhanced cross-reactive neutralizing response.

### SUMMARY OF THE INVENTION

The invention encompasses a vaccine and/or immunogenic composition comprising an isolated HIV envelope protein capable of inducing the production of a cross-reactive neutralising anti-serum against multiple strains of HIV-1 in vitro wherein the V3 region of the HIV envelope protein comprises amino acids 313 to 325 of SEQ ID NO: 1 or immunogenic fragments thereof; and a Toll-like receptor (TLR) 4 ligand, in combination with a saponin.

In a further embodiment of the present invention is provided a vaccine and/or immunogenic composition comprising an isolated HIV envelope protein capable of inducing the production of a cross-reactive neutralising anti-serum against multiple strains of HIV-1 in vitro wherein HIV envelope protein comprises an amino acid sequence with at least 92% identity to SEQ ID NO: 1; and a Toll-like receptor (TLR) 4 ligand, in combination with a saponin.

In some embodiments, the Toll-like receptor (TLR) 4 ligand is a lipid A derivative including, but not limited to, monophosphoryl lipid A. Examples of monophosphoryl lipid A include, but are not limited to, 3 Deacylated monophosphoryl lipid A (3 D-MPL). In some embodiments, the saponin is QS-21. In some embodiments, the saponin is presented in the form of a liposome, ISCOM or an oil in water emulsion.

In some embodiments of the vaccine and/or immunogenic composition, the HIV envelope protein comprises an amino acid sequence with at least ninety two percent, at least ninety five percent, or at least ninety-nine percent sequence identity to SEQ ID NO: 1. In some embodiments, the HIV envelope protein comprises the amino acid sequence of SEQ ID NO: 1.

The invention encompasses a method of inducing an immune response by administration of any of the aforementioned vaccine and/or immunogenic compositions to a human in need thereof. The invention encompasses the use of the aforementioned vaccine and/or immunogenic compositions in the manufacture of a medicament for the prevention and/or treatment of HIV infection and Acquired Immune Deficiency Syndrome (AIDS).

### BRIEF DESCRIPTION OF THE DRAWINGS

The foregoing summary, as well as the following detailed description of the invention, will be better understood when read in conjunction with the appended figures. For the purpose of illustrating the invention, shown in the figures are embodiments of the present invention. It should be understood, however, that the invention is not limited to the precise arrangements, examples, and instrumentalities shown.

FIG. 1: Inhibition of HIV-1 virus pseudotyped with envelope glycoproteins of various strains. Three rabbits each received HIV-1 R2 strain env protein, gp120 or gp140, in adjuvant A, or adjuvant A alone. Sera were collected after three or four doses and tested in triplicate at 1:5 dilutions for neutralization of HIV-1 pseudotypes. Mean luminescence for the three control sera against each virus was calculated. Percent inhibition was calculated for each immune and control serum by comparison to the mean for the control sera. In FIG. 1, solid circles indicate the results from individual sera and horizontal bars indicate means and standard deviations of the pooled sera.

FIG. 2: Neutralization titers of sera from rabbits. Sera were collected from rabbits after 3 or 4 doses of R2 gp120 or gp140 in adjuvant A and used in a neutralization assay as described below. Endpoints were determined as the last dilution inhibiting luminescence to less that 50% of the level observed on average for virus cultured in the presence of the same dilution of pooled sera from concurrent control rabbits. Results are shown for each individual immune serum against the various strains of HIV-1 tested based on triplicate determinations with geometric means and standards deviations.

FIG. 3: Neutralization of wild type and mutant strains of R2 and 14/00/4. Viruses were pseudotyped with glycoproteins from wild type strain R2, wild type strain 14/00/4, mutant strain R2 (313-4PM/HI) or mutant strain 14/00/4 (162T/A). Sera collected from rabbits after three doses of R2 gp120 or gp140 in adjuvant A were used in a neutralization assay, as described below, with the abovementioned strains. Titers were determined as described for FIG. 2. Wild type strains R2 and 14/00/4 are represented by closed circles and the corresponding mutant strains R2 (313-4PM/HI) and 14/00/4 (162T/A) are represented by open circles. Results are shown for individual sera (circles), geometric means (bars) and standard deviations. The numbers shown above sets of data points indicate probabilities by student t test comparing neutralization of the wild-type and mutant strains.

FIG. 4: Comparative neutralization of pathogenic SHIV and HIV strains. Viruses were pseudotyped with envelope glycoproteins from pathogenic SHIV and HIV strains DH12, SF162 and 89.6. Sera collected from rabbits after three doses of R2 gp120 or gp140 in adjuvant A were used in neutralization assays, as previously described, with the abovementioned strains. Titers were calculated as described in FIG. 2. HIV strains are represented by closed circles and matched pathogenic SHIV strains are represented by open circles.

FIG. 5: Antibodies obtained from immunized rabbit sera bind to gp140 of pathogenic HIV strains. Sera collected from rabbits immunized with gp120 or gp140 were tested for antibody binding to gp140 from strains R2, 14/00/4, and CM243. Sera obtained after both third and forth immunization was assayed using an enzyme-linked immunoassay. Optical densities obtained that were greater than twice background were considered positive for antibody binding. Endpoints were calculated by regression analysis.

FIG. 6: Comparative inhibition of HIV-1 infection of HOS-CD4+CCR5+ cell cultures by sera from gp120_{R2} and gp140_{R2} immunized rabbits, as manifest by levels of luciferase reporter gene expression. The viruses were pseudotyped with envelope glycoproteins of the HIV-1 strains and subtypes indicated. Viruses were incubated in the presence of 1:5 diluted test or control sera prior to cell culture inoculation. Mean luminescence after infection in the presence of control sera was calculated. Luminescence obtained in the presence of individual test and control sera was calculated and used to determine percent inhibition in comparison to the control mean. Percent inhibition by individual control sera is shown to illustrate the variance observed.

FIG. 7: Potent neutralization of strains sensitive to gp120-induced antibodies develops after a brief immunization regimen. Shown are rates of development of neutralizing antibody responses after immunization of rabbits with gp120 (closed diamond) or gp140 (closed circle) in AS02A adjuvant, compared to rabbits given adjuvant alone (open square). Sera from rabbits immunized with either gp120 or gp140, while sera from rabbits immunized with gp140 only neutralized strains DU151-2, SVPB4, and SVPB12 neutralized the strains R2, SF162, MACS4, SVPB9, and 14/00/4. Percent inhibition was calculated as described in FIG. 6. Sera were collected for testing 10 days after each dose of immunogen given at weeks 0, 3, 6, and 28.

FIG. 8: Antibodies induced by gp140 neutralize pathogenic SHIV and parent strains of HIV-1 from which they were derived. Serial dilutions of sera from rabbits taken after three or four doses of immunogen were compared to serial dilutions of pooled sera from rabbits given adjuvant only. The neutralization endpoint was assigned as the highest dilution of test serum that resulted in ≧50% inhibition of luminescence compared to the same dilution of control serum.

FIG. 9: Neutralization endpoint titers of sera from gp120_{R2} and gp140_{R2} immunized rabbits against various strains of HIV-1. Results are shown for sera obtained post 3 or 4 doses of immunogen. Sera that inhibited <50% were assigned titers <1:5. Sera that inhibited ≧50-74% were assigned titers of 1:5. Sera that inhibited ≧75% were tested for neutralization endpoints. Serial dilutions of test sera were compared to serial dilutions of pooled, concurrent control sera. The endpoint was considered to be the highest dilution that resulted in ≧50% or ≧75% inhibition of luminescence compared to the same dilution of the control serum pool.

FIG. 10: HIV-1 Specificity of Neutralizing Antibody Responses. FIG. 10A shows that sera from gp120_{R2}- and gp140_{R2}-immunized rabbits do not neutralize HIV-2 Env and VSV G pseudotyped viruses. Rabbit sera obtained after four doses of gp120_{R2} or gp140_{R2} (both open circles and dashed lines) and pooled concurrent control sera (closed circles) were tested in triplicate at serial dilutions beginning at 1:5. FIGS. 10B and 10C show that extensive absorption of gp140-immune rabbit sera with 293T cells does not deplete primary HIV-1 neutralizing activity. FIG. 10B shows the results of a FACS analysis of sera from rabbits 4 (closed triangle), 5 (closed square), and 6 (closed circle) post fourth dose gp140 and pooled prebleed sera from the same rabbits (closed diamond) before and after one, two, or three consecutive absorptions with 293T cells. Percent positive cells compared to negative control results obtained using PBS and goat serum without rabbit sera are shown. FIG. 10C shows the inhibition of neutralization resistant subtype B (SVPB11) and C (DU123) strains of HIV-1 by post fourth dose serum from Rabbit 4 (open symbols), in comparison to pooled sera from the control rabbits (solid symbols) at the same time point, before (closed square, open square) and after (closed circle, open circle) three consecutive absorptions with 293T cells. Standard deviations are shown in relation to each data point. FIG. 10D shows that neutralizing activity in serum is IgG mediated. IgG was purified from post sixth dose sera from Rabbit 4 and from control rabbits, and tested in comparison to the same sera for neutralization. Results obtained using IgG are shown as closed symbols, and using sera as open symbols. Results obtained using immune sera and IgG are shown as solid lines, while results obtained using control IgG are shown as dashed lines. Neutralization of R2 virus by IgG (closed triangle) and serum (open triangle) was essentially identical. The five addition subtype B strains tested (upper panel) were SVPB5, SVPB11, SVPB14, SVPB16, and SVPB19. The remaining strains (subtypes) were DU422 (C), DU165.12 (C), UG273 (A), NYU1545 (D), and CM243 (E) (lower panel).

FIG. 11: Reactivity of sera and IgG from gp140-immunized rabbits with 293T cells is removed by absorption with 293T cells. Sera were collected after the sixth gp140 or control immunization, IgG was purified and IgG were subjected to three serial absorptions with 293T cells. Immune (closed square) and control (open square) sera diluted 1:200 or 1:1000 and immune (closed circle) and control (open circle) IgG at 50 or 10 ng/ml were tested by FACS analysis for binding to 293T cells, as described in FIG. 10.

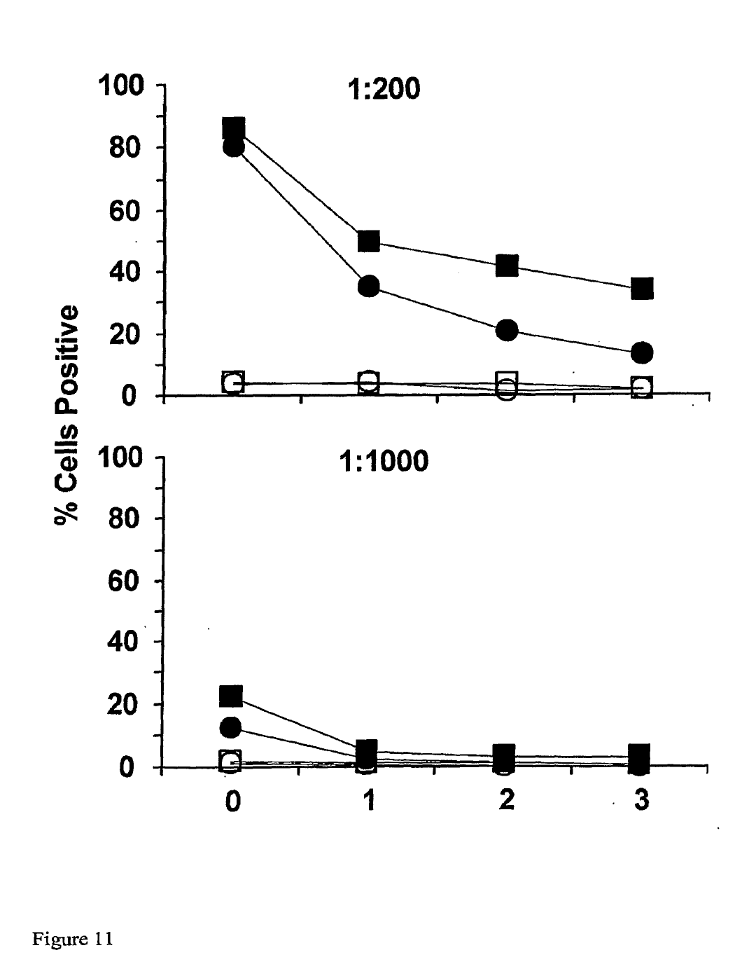

FIG. 12: IgG from rabbits after six doses of gp140 mediates HIV-1-specific neutralization. Sera (squares) and IgG (circles) from immune (closed square, closed circle) and control (open square, open circle) rabbits was subjected absorbed with 293T cells, as shown in FIG. 11. Absorbed and unabsorbed sera and IgG were compared for neutralization of VSV and the HIV-1 strains SVPB19 and DU422. Unabsorbed immune sera and IgG achieved ≧50% neutralization of VSV at 1:10 and 1:20 dilutions, respectively, while absorbed serum achieved neutralization at 1:5 dilution only, and absorbed IgG did not neutralize VSV. Standard deviations around individual data points are shown.

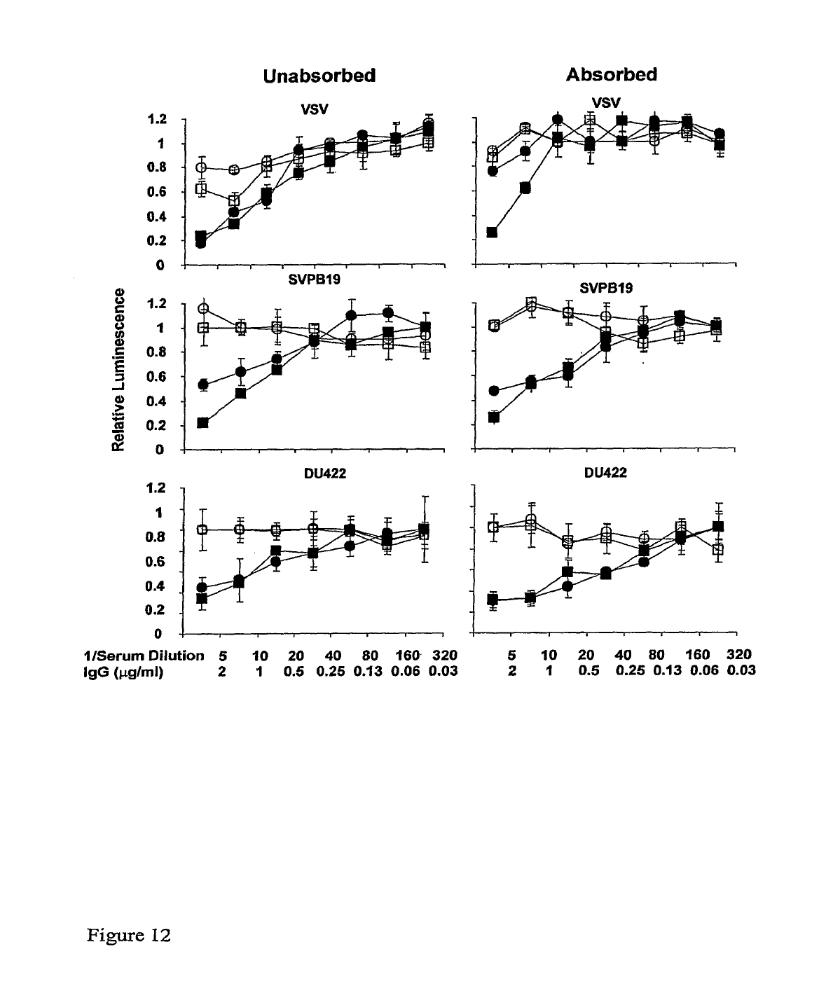

FIG. 13: Antibodies with greater strain specificity are induced by gp120 than gp140. ELISA was conducted using gp140s from the HIV-1 strains R2, 14/00/4 (subtype F), and CM243 (subtype E). Sera were tested in serial two-fold dilutions beginning at 1:200, and sera that were negative at that dilution were assigned titers of 1:100 for calculation of geometric mean titers and presentation.

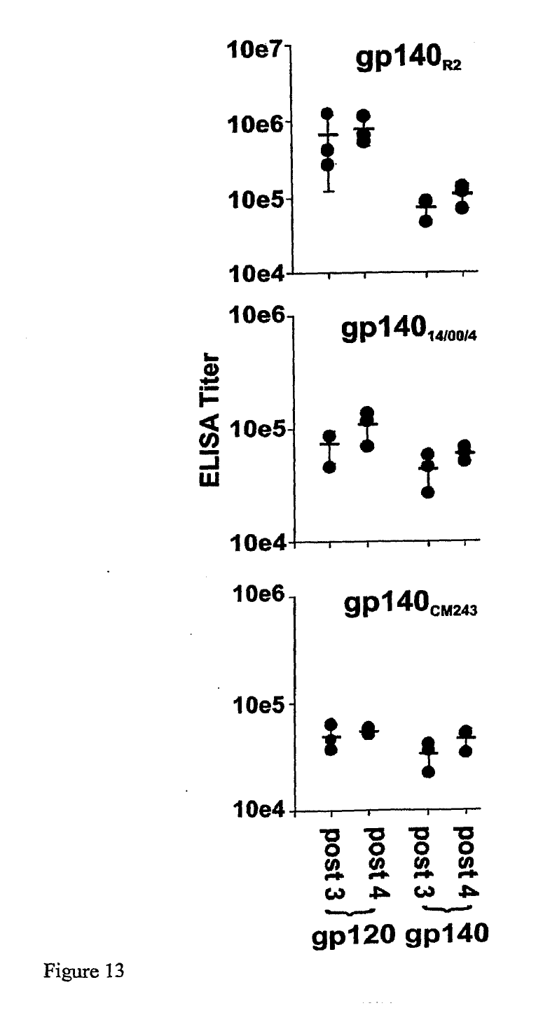

### DETAILED DESCRIPTION

All cited patents, patent applications, publications and other documents cited in this application are herein incorporated by reference in their entirety. The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims.

A goat of immunization against HIV is to induce neutralizing antibody (NA) responses broadly reactive against diverse strains of virus. The present inventors found that immunization of rabbits with oligomeric gp140 from the HIV-1 strain R2 adjuvanted with certain adjuvants, results in induction of potent, broadly cross-reactive neutralizing antibody responses. Sera from animals immunized with gp140 inhibited infectivity of viruses pseudotyped with each of 45 different strains of HIV-1 envelope glycoprotein. The strains included 19 subtype B strains, 14 subtype C strains, and subtype A, D, AE, F, AG, H, and complex CRF envelopes. The results constitute the first demonstration of an HIV-1 neutralizing response to immunization that is truly broadly cross-reactive, provides new principles for design of non-human primate immunization and challenge studies, and establishes a model system for dissecting the basis for highly cross-reactive neutralization of HIV-1. The present invention encompasses vaccine and immunogenic compositions, methods of inducing an immune response using the provided compositions and the use of the compositions of the invention in the manufacture of a medicament for the prevention and/or treatment of HIV infection and AIDS.

Although any methods and materials similar or equivalent to those described herein can be used in the practice or testing of the present invention, examples of suitable methods and materials are described. Unless defined otherwise, all technical and scientific terms used herein have the same meaning as commonly understood by one of ordinary skill in the art to which this invention belongs.

A meaning for “identity” for polypeptides, are provided as follows. Polypeptide embodiments further include an isolated polypeptide comprising a polypeptide having at least a 80, 85, 90, 91, 92, 93, 94, 95, 96, 97, 98, 99 or 100% identity to a polypeptide reference sequence, wherein said polypeptide sequence may be identical to the reference sequence or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids by the integer defining the percent identity divided by 100 and then subtracting that product from said total number of amino acids, or: na xa−(xa y), wherein na is the number of amino acid alterations, xa is the total number of amino acids in the sequence, y is 0.95 for 95%, 0.97 for 97% or 1.00 for 100%, and is the symbol for the multiplication operator, and wherein any non-integer product of xa and y is rounded down to the nearest integer prior to subtracting it from xa.

Homology or sequence identity at the nucleotide or amino acid sequence level can also be determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell.

“Polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers, and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure and Molecular Properties, 2nd Ed., Creighton (Ed.), W. H. Freeman and Company, New York (1993) and Wold, Posttranslational Protein Modifications: Perspectives and Prospects, pp. 1-12 in Posttranslational Covalent Modification of Proteins, Johnson, Ed., Academic Press, New York (1983); Seifter et al. (1990) Meth. Enzymol. 182, 626-646 and Rattan et al. (1992) Ann. N.Y. Acad. Sci. 663, 48-62. Polypeptides may be branched or cyclic, with or without branching. Cyclic, branched and branched circular polypeptides may result from post-translational natural processes and may be made by entirely synthetic methods, as well.

Vaccine and/or Immunogenic Compositions

A vaccine and/or immunogenic composition of the present invention induces at least one of a number of humoral and/or cellular immune responses in a human who has been administered the composition or is effective in enhancing at least one immune response against at least one strain of HIV, such that the administration is suitable for vaccination purposes and/or prevention of HIV infection by one or more strains of HIV-1. The composition of the present invention delivers to a subject in need thereof a recombinant env protein, comprising gp120, gp140, and/or gp160 from one or more HIV-1 and an adjuvant. In some embodiments, the gp120 and gp140 are from HIV-1 strain R2 as described in WO 00/07631.

In some embodiments, the vaccine and/or immunogenic composition comprises one or more HIV-1 envelope proteins as described herein. Envelope proteins of the invention include the full length envelope protein with an amino acid sequence comprising SEQ ID NO: 1, gp120 comprising the amino acid sequence corresponding to amino acids 1 to 520 of SEQ ID NO: 1, gp41 comprising the amino acid sequence corresponding to amino acids 521 to 866 of SEQ ID NO: 1, as well as polypeptides and peptides corresponding to the V3 domain and other domains such as V1/V2, C3, V4, C4 and V5. These domains correspond to the following amino acid residues of SEQ ID NO: 1.

| Domain | Amino Acid |
|---|---|
| C1 | 30 to 124 |
| V1 | 125 to 162 |
| V2 | 163 to 201 |
| C2 | 202 to 300 |
| V3 | 301 to 336 |
| C3 | 337 to 387 |
| V4 | 388 to 424 |
| C4 | 425 to 465 |
| V5 | 466 to 509 |
| C5 | 510 to 520 |

The compositions of the invention may contain proteins and/or polypeptides comprising any single domain and may be of variable length but include the amino acid residues 313 to 325 of SEQ ID NO: 1 which differ from previously sequenced envelope proteins. For instance, peptides of the invention which include all or part of the V3 domain may comprise the sequence: PM X₁ X₂ X₃ X₄ X₅ X₆ X₇ X₈ X₉ X₁₀ Q, wherein X₁ to X₁₀ are any natural or non-natural amino acids (P refers to Proline, M refers to methionine and Q refers to Glutamine). In one embodiment of the present invention, envelope proteins of the invention are at least about 90, 91, 92, 93, 94, 95, 96, 97, 98, or 99% identical to the V3 region of the HIV envelope protein of SEQ ID NO: 1 (amino acids 301 to 336). Accordingly, V3 peptides of the invention comprise about 13 amino acids but may be at least 14, 15, 16, 17, 18, 19, 20, 21, 22, 23, 24, 25, 30, 35, 36, 37, 38, 39, 40, 45, 50 or more amino acids in length. In one embodiment, a V3 domain comprises the amino acid sequence PMGPGRAFYTTGQ (amino acids 313 to 325 of SEQ ID NO: 1) (SEQ ID NO: 2).

In another embodiment of the invention, envelope proteins comprising all or part of the V1/V2 domain comprise an amino acid sequence with an alanine residue at a position corresponding to amino acid 167 of SEQ ID NO: 1. For instance, peptides of the invention spanning the V1/V2 domain may comprise the amino acid sequence FNIATSIG (amino acids 164 to 171 of SEQ ID NO: 1) (SEQ ID NO: 3) and may be about 8, 9, 10, 15, 20, 25, 30, 35, 40, 45, 50 or more amino acids in length. As used herein, “at a position corresponding to” refers to amino acid positions in HIV envelope proteins or peptides of the invention which are equivalent to a given amino acid residue in the sequence of SEQ ID NO: 1 in the context of the surrounding residues or by alignment of particular sequences.

In the present invention, the vaccine and/or immunogenic composition comprises an adjuvant. As used herein, “adjuvant” refers to an agent which, while not having any specific antigenic effect in itself, may stimulate the immune system, increasing the response to a vaccine. In some embodiments, the adjuvant comprises a Toll like receptor (TLR) 4 ligand, in combination with a saponin. The Toll like receptor (TLR) 4 ligand may be for example, an agonist such as a lipid A derivative particularly monophosphoryl lipid A or more particularly 3 Deacylated monophosphoryl lipid A (3 D-MPL). 3 D-MPL is sold under the trademark MPL® by Corixa Corporation and primarily promotes CD4+ T cell responses with an IFN-g (Th1) phenotype. It can be produced according to the methods disclosed in GB 2220211A. Chemically, it is a mixture of 3-deacylated monophosphoryl lipid A with 3, 4, 5 or 6 acylated chains. In one embodiment in the compositions of the present invention small particle 3 D-MPL is used. Small particle 3 D-MPL has a particle size such that it may be sterile-filtered through a 0.22 μm filter. Such preparations are described in WO 94/21292.

The adjuvant may also comprise one or more synthetic derivatives of lipid A which are known to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate) as described in WO 95/14026.

OM 294 DP (3S,9R)-3-[(R)-dodecanoyloxytetradecanoylamino]-4-oxo-5-aza-9(R)—[(R)-3-hydroxytetradecanoylamino]decan-1,10-diol,1,10-bis(dihydrogenophosphate) as described in WO 99/64301 and WO 00/0462.

OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) as described in WO 01/46127.

Other TLR4 ligands which may be used include, but are not limited to, alkyl Glucosaminide phosphates (AGPs) such as those disclosed in WO 98/50399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both can be used as one or more adjuvants in the compositions of the invention.

A preferred saponin for use in the present invention is Quil A and its derivatives. Quil A is a saponin preparation isolated from the South American tree Quillaja Saponaria Molina and was first described as having adjuvant activity by Dalsgaard et al. (1974) Saponin adjuvants, Archiv. für die gesamte Virusforschung, Vol. 44, Springer Verlag, pp. 243-254. Purified fragments of Quil A have been isolated by HPLC which retain adjuvant activity without the toxicity associated with Quil A (EP 0 362 278), for example QS7 and QS21 (also known as QA7 and QA21). QS21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly preferred, these formulations further comprise a sterol (WO 96/33739). The saponins forming part of the present invention may be separate in the form of micelles, mixed micelles (preferentially, but not exclusively with bile salts) or may be in the form of ISCOM matrices (EP 0 109 942 B1), liposomes or related colloidal structures such as worm-like or ring-like multimeric complexes or lipidic/layered structures and lamellae when formulated with cholesterol and lipid, or in the form of an oil in water emulsion (for example as in WO 95/17210). The saponins may be associated with a metallic salt, such as aluminum hydroxide or aluminum phosphate (WO 98/15287). In some embodiments, the saponin is presented in the form of a liposome, ISCOM or an oil in water emulsion.

In some embodiments, adjuvants are combinations of 3D-MPL and QS21 (EP 0671948 B1) and oil in water emulsions comprising 3D-MPL and QS21 (WO 95/17210, WO 98/56414).

In one embodiment of the present invention is provided an immunogenic composition comprising an isolated HIV envelope protein capable of inducing the production of a cross-reactive neutralising anti-serum against multiple strains of HIV-1 in vitro wherein the V3 region of the HIV envelope protein comprises amino acids 313 to 325 of SEQ ID NO: 1; and an adjuvant comprising an oil in water emulsion with QS21 and MPL which may also have tocopherol present, for example wherein the emulsion contains: 5% Squalene, 5% tocopherol, 2.0% Tween 80, and which may have a particle size of approximately 180 nm. Alternatively, the adjuvant may comprise liposomal QS21 and MPL, for example, wherein the liposomes have a size of approximately 100 nm and are referred to as SUV (for small unilamelar vesicles).

In a further embodiment of the present invention is provided an immunogenic composition comprising an isolated HIV envelope protein capable of inducing the production of a cross-reactive neutralising anti-serum against multiple strains of HIV-1 in vitro wherein the HIV envelope protein comprises an amino acid sequence with at least 92% identity to SEQ ID NO: 1, for example 93%, 94%, 95%, 96%, 97%, 98%, 99% or 100% identity to SEQ ID NO: 1; and an adjuvant comprising QS21 and MPL which may also have tocopherol present, for example, wherein the emulsion contains: 5% Squalene, 5% tocopherol, 2.0% Tween 80, and which may have a particle size of approximately 180 nm. Alternatively, the adjuvant may comprise liposomal QS21 and MPL, for example, wherein the liposomes have a size of approximately 100 nm and are referred to as SUV (for small unilamelar vesicles).

In yet a further embodiment of the present invention is provided an immunogenic composition comprising an isolated HIV envelope protein capable of inducing the production of a cross-reactive neutralising anti-serum against multiple strains of HIV-1 in vitro wherein the HIV envelope protein consists of an amino acid sequence of SEQ ID NO: 1; and an adjuvant comprising an oil in water emulsion with QS21 and MPL which may also have tocopherol present, for example wherein the emulsion contains: 5% Squalene, 5% tocopherol, 2.0% Tween 80, and which may have a particle size of approximately 180 nm. Alternatively, the adjuvant may comprise liposomal QS21 and MPL, for example, wherein the liposomes have a size of approximately 100 nm and are referred to as SUV (for small unilamelar vesicles).

Immunogenic fragments as described herein will contain at least one epitope of the antigen and display HIV antigenicity and are capable of raising an immune response when presented in a suitable construct, such as for example when fused to other HIV antigens or presented on a carrier, the immune response being directed against the native antigen. In one embodiment of the present invention, the immunogenic fragments contain at least 20 contiguous amino acids from the HIV antigen, for example, at least 50, 75, or 100 contiguous amino acids from the HIV antigen.

In one embodiment of the invention, the vaccine and/or immunogenic composition comprises the adjuvant AS02A (GlaxoSmithKline Biologicals, Rixensart, Belgium). In another embodiment, the vaccine and/of immunogenic composition comprises the adjuvant AS03A (GlaxoSmithKline Biologicals, Rixensart, Belgium).

In another embodiment of the invention, the vaccine and or/immunogenic compositions may be part of a pharmaceutical composition. The pharmaceutical compositions of the present invention may contain suitable pharmaceutically acceptable carriers comprising excipients and auxiliaries that facilitate processing of the active compounds into preparations that can be used pharmaceutically for delivery to the site of action.

The vaccine and/or immunogenic compositions of the present invention may further comprise additional HIV-1 env proteins that may correspond to gp120 and gp140 from different strains that may further potentiate the immunization methods of the invention.

### Methods of Use

The invention encompasses methods of preventing and/or treating HIV infection and/or AIDS comprising administering the compositions of the invention. Active immunity elicited by vaccination with an HIV-1 env proteins gp120 and/or gp140 with the adjuvants described herein can prime or boost a cellular or humoral immune response. An effective amount of the HIV-1 env protein, gp120 and/or gp140, or antigenic fragments thereof, can be prepared in an admixture with an adjuvant to prepare a vaccine.

The administration of a vaccine and/or immunogenic composition comprising or encoding for HIV-1 env proteins, gp120 and/or gp140 with one or more adjuvants described herein, can be for either a “prophylactic” or “therapeutic” purpose. In one aspect of the present invention, the composition is useful for prophylactic purposes. When provided prophylactically, the vaccine composition is provided in advance of any detection or symptom of HIV infection or AIDS. The prophylactic administration of an effective amount of the compound(s) serves to prevent or attenuate any subsequent HIV infection. When provided therapeutically, the vaccine is provided in an effective amount upon the detection of a symptom of actual infection. A composition is said to be “pharmacologically acceptable” if its administration can be tolerated by a recipient patient. Such an agent is said to be administered in a “therapeutically or prophylactically effective amount” if the amount administered is physiologically significant. A vaccine or immunogenic composition of the present invention is physiologically significant if its presence results in a detectable change in the physiology of a recipient patient, for example, by enhancing a broadly reactive humoral or cellular immune response to one or more strains of HIV-1. The “protection” provided need not be absolute (i.e., the HIV infection or AIDS need not be totally prevented or eradicated), provided that there is a statistically significant improvement relative to a control population. Protection can be limited to mitigating the severity or rapidity of onset of symptoms of the disease.

A vaccine or immunogenic composition of the present invention can confer resistance to multiple strains of HIV-1. The present invention thus concerns and provides a means for preventing or attenuating infection by at least two HIV-1 strains. As used herein, a vaccine is said to prevent or attenuate a disease if its administration to an individual results either in the total or partial attenuation (i.e., suppression) of a symptom or condition of the disease, or in the total or partial immunity of the individual to the disease.

At least one vaccine of the present invention can be administered by any means that achieve the intended purpose, using e.g. a pharmaceutical composition as described herein. For example, administration of such a composition can be by various parenteral routes such as subcutaneous, intravenous, intradermal, intramuscular, intraperitoneal, intranasal, transdermal, or buccal routes. In one embodiment of the present invention, the composition is administered by subcutaneously. Parenteral administration can be by bolus injection or by gradual perfusion over time.

A typical regimen for preventing, suppressing, or treating a disease or condition which can be alleviated by a cellular immune response by active specific cellular immunotherapy, comprises administration of an effective amount of a vaccine composition as described above, administered as a single treatment, or repeated as enhancing or booster dosages, over a period up to and including one week to about twenty-four months.

According to the present invention, an “effective amount” of a vaccine composition is one which is sufficient to achieve a desired biological effect, in this case at least one of cellular or humoral immune response to one or more strains of HIV-1. It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent examples of dose ranges which may be suitable for administering compositions of the present invention. However, the dosage may be tailored to the individual subject, as is understood and determinable by one of skill in the art, without undue experimentation (see, for example, Beers (1999) Merck Manual of Diagnosis and Therapy, Merck & Company Press; Gennaro et al. (2005), Goodman & Gilman's The Pharmacological Basis of Therapeutics, McGraw-Hill; Katzung (1988) Clinical Pharmacology, Appleton & Lange; which references and references cited therein, are entirely incorporated herein by reference).

The invention further provides methods of preparing the polypeptides described herein which method comprises expressing a polynucleotide encoding the polypeptide in a suitable expression system, particularly a prokaryotic system such as E. coli and recovering the expressed polypeptide. Preferably, expression is induced at a low temperature, which is a temperature below 37°, to promote the solubility of the polypeptide.

The invention further provides a process for purifying a polypeptide as described herein, which process comprises:

i. Providing a composition comprising the unpurified polypeptide;

ii. Subjecting the composition to at least two chromatographic steps;

iii. Optionally carboxyamidating the polypeptide; and

iv. Performing a buffer exchange step to provide the protein in a suitable buffer for a pharmaceutical formulation.

The carboxyamidation may be performed between the two chromatographic steps. The carboxyamidation step may be performed using iodoacetimide. In one example, the process according to the invention uses no more than two chromatographic steps.

The invention further provides pharmaceutical compositions and immunogenic compositions and vaccines comprising the polypeptides and adjuvant combinations according to the invention, in combination with a pharmaceutically acceptable adjuvant or carrier.

Vaccines according to the invention may be used for prophylactic or therapeutic immunization against HIV. The invention further provides the use of the polypeptide compositions as described herein, in the manufacture of a vaccine for prophylactic or therapeutic immunization against HIV.

The vaccine of the present invention will contain an immunoprotective or immunotherapeutic quantity of the polypeptide and adjuvant combination and may be prepared by conventional techniques.

Vaccine preparation is generally described in New Trends and Developments in Vaccines, edited by Voller et al. (1978), University Park Press, Baltimore, Md. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in the vaccine dose is selected as an amount which induces an immunoprotective response without significant, adverse side effects in typical vaccinees. Such amount will vary depending upon which specific immunogen/adjuvant combination is employed and the vaccination regimen that is selected. Generally, it is expected that each dose will comprise 1 to 1000 μg of each protein, for example, 2 to 200 μg, or 4 to 40 μg of the polypeptide. An optimal amount for a particular vaccine can be ascertained by standard studies involving observation of antibody titres and other immune responses in subjects. Following an initial vaccination, subjects may receive a subsequent boosting dose. Such a boosting dose may be administered in about 4 weeks following the initial vaccination, and a subsequent second booster immunization.

These dosages can be suspended in any appropriate pharmaceutical vehicle or carrier in sufficient volume to carry the dosage. Generally, the final volume, including carriers, adjuvants, and the like, typically will be at least 0.1 ml, more typically at least about 0.2 ml. The upper limit is governed by the practicality of the amount to be administered, generally no more than about 0.5 ml to about 1.0 ml.

The recipients of the vaccines of the present invention can be any mammal which can acquire specific immunity via a cellular or humoral immune response to HIV-1, where the cellular response is mediated by an MHC class I or class II protein. Among mammals, the recipients may be mammals of the Orders Primata (including humans, chimpanzees, apes and monkeys). In one embodiment of the present invention there is provided a method of treating humans with immunogenic compositions of the invention. The subjects may be infected with HIV or provide a model of HIV-1 infection (see, for example, Hu et al. (1987) Nature 328, 721-723, which reference is entirely incorporated herein by reference).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein. The following materials and methods are provided with respect to the subsequent examples but do not limit the multiplicity of materials and methodologies encompassed by the present invention.

The following examples utilize Env derived from an HIV-1 infected individual whose serum antibodies exhibit extensive neutralizing cross-reactivity against many primary strains of HIV-1 of diverse virus subtypes (Dong et al. (2003) J. Virol. 77, 3119-3130; Zhang et al. (2002) J. Virol. 76, 644-655). This Env, designated R2, is highly unusual as a naturally occurring HIV-1 envelope that is be capable of mediating CD4-independent infection (Zhang et al. (2002) J. Virol. 76, 644-655). In immunogenicity studies conducted in small animals and non-human primates, it was demonstrated that this Env induces neutralizing antibodies against multiple HIV-1 strains, and in non-human primates induction of protection against intravenous challenge with a heterologous strain of Simian-Human Immunodeficiency Virus (SHIV) has been shown (Dong et al. (2003) J. Virol. 77, 3119-3130; Quinnan et al. (2005) J. Virol. 79, 3358-3369).

Production of gp140 and gp120. The gp140_{R2}, gp140_{14/00/4}, and gp140_{CM243} coding sequences were prepared by insertion of two translational termination codons just prior to the predicted gp41 transmembrane region and arginine to serine substitutions at to disrupt protease cleavage signals to increase the yield of oligomeric envelope glycoprotein during production (Dong et al. (2003) J. Virol. 77, 3119-3130; Quinnan et al. (2005) J. Virol. 79, 3358-3369). The gp120_{R2} coding sequence was prepared by insertion of a translational termination codon. The genes were subcloned into the vaccinia vector, pMCO2 (Doug et al. (2003) J. Virol. 77, 3119-3130). Recombinant vaccinia viruses were generated using standard methodology (Dong et al. (2003) J. Virol. 77, 3119-3130; Quinnan et al. (2005) J. Virol. 79, 3358-3369). Glycoproteins were produced and purified from culture supernatants, prepared with serum-free media, using lentil lectin Sepharose 4B affinity, followed by size exclusion chromatography (Dong et al. (2003) J. Virol. 77, 3119-3130; Quinnan et al. (2005) J. Virol. 79, 3358-3369). The oligomeric gp140_{R2} has been extensively analyzed, and has been shown based on size exclusion chromatography to be approximately 40% trimer and 60% dimer (Dong et al. (2003) J. Virol. 77, 3119-3130; Quinnan et al. (2005) J. Virol. 79, 3358-3369). Analysis by SDS-PAGE and commassie blue staining revealed electrophoretic migration typical of glycoprotein, and purity of 98%. Endotoxin concentration was 0.2-1.1 EU/μg.

Virus strains. Envelope gene encoding plasmids utilized for preparation of pseudotyped viruses used in this study are described in Table 1. The plasmids beginning with the letters SVPB or DU encode envelope glycoproteins of subtypes B and C considered representative of current epidemic strains (Li et al. (2005) J. Virol. 79, 10108-10125) are listed in Table 1. All are from primary viruses. The subtype B strains and three of the subtype C strains from Dr. Montefiori are included in panels he has provided to NIH. These strains were selected on the basis of being representative of the epidemic and resistant to neutralization by sera from individuals infected with strains of the same subtypes. The env clones from individuals from Xinjiang, China, have not been previously described. The results of neutralization of these strains by sera from subtype C infected individuals from Xinjiang are shown in Table 2. The strains 5-4, 6-15, 7-102, 8-145, and 10-35 were all resistant to neutralization by most or all heterologous sera tested. Strains 1-27 and 9-26, which were among those that were sensitive to neutralization by gp120-induced antibodies in the present study, were among those that were relatively more sensitive to neutralization by the sera from HIV-1 infected individuals from Xinjiang. The remaining strains were cloned at various times in our laboratory and are described in the noted publications (Zhang et al. (2002) J. Virol. 76, 644-655; Zhang et al. (1999) J. Virol. 73, 5225-5230; Dong et al. (2003) J. Virol. 77, 3119-3130; Quinnan et al. (2005) J. Virol. 79, 3358-3369; Quinnan et al. (1999) AIDS Res. Hum. Retrovir. 15, 561-570; Quinnan et al. (1998) AIDS Res. Hum. Retrovir. 14, 939-949; Cham et al. (2005) Virology). The characterization of the strains was based on testing in a pseudotyped virus assay, similar to the one used in this study.

The strains GXE14, 24/00/4, 14/00/4, CA1, VI423, NYU1026, NYU1423, GXE14, and VI1793 were described by Cham et al. (Cham et al. (2005) Virology). The strains 24/00/4, 14/00/4, VI 423, and CA1 were sensitive to neutralization by human sera tested, while the strains NYU1026, NYU1423, GXE14, and VI1793 were resistant. The strains MACS4 and MACS9 were described by Zhang et al. (Zhang et al. (1999) J. Virol. 73, 5225-5230). The MACS4 strain was sensitive to neutralization by sera from the majority of sera from Multicenter AIDS Cohort Study participants tested, while MACS9 was not. The CM243 strain is generally resistant to neutralization by sera from non-subtype E infected individuals. The strain VI 525 is resistant to most human sera with the exception of sera with broad cross-reactivity (Beirnaert et al. (2001) Virology 281, 305-314). Little information is available regarding the sensitivity of the strains UG273 and NYU1545 to human serum.

TABLE 1
Virus strains used in pseudotyped virus neutralization assays

| Subtype | Strain | Source | Comment |
|---|---|---|---|
| A | VI525-1 | Africa | Beirnaert et al. (2000) J. Med. Virol. |
| | | | 62, 14-24; Igarahi (1999) Proc. Natal. |
| | | | Acad. Sic. USA 96, 14049-14054; |
| | | | Beirnaert et al. (2001) Virology 281, |
| | | | 305-314. |
| | UG273 | Uganda | Cham et al. (2006) Virology 347, 36-51. |
| | NYU1423 | Cameroon | |
| | NYU1026A | Cameroon | |
| B | R2 | U.S. | Quinnan et al. (1999) AIDS Res. |
| | | | Human. Retrovir. 15, 561-570; Zhang |
| | | | et al. (2002) J. Virol. 76, 644-655. |
| | SF162 | U.S. | Zhang et al. (1999) J. Virol. 73, 5225-5230. |
| | SHIV-SF162P3 | U.S. | Quinnan et al. (2005) J. Virol. 79, |
| | 89.6 | U.S. | 3358-3369. |
| | SHIV-89.6p | U.S. | |
| | DH12 | U.S. | |
| | SHIV-DH12R | U.S. | |
| | Clone7 | | |
| | MACS4 | U.S. | Zhang et al. (1999) J. Virol. 73, 5225-5230; |
| | MACS9 | U.S. | Quinnan et al. (1998) AIDS Res. |
| | | | Human. Retrovir. 14, 939-949. |
| | VI1423 | Belgium | Beirnaert et al. (2000), J. Med. Virol. |
| | | | 62, 14-24; Beirnaert et al. (2001) |
| | | | Virology 281, 305-314. |
| | SVPB1 | U.S. | Mascola et al. (2005) J. Virol. 79, |
| | SVPB2 | Not provided | 10103-10107. |
| | SVPB3 | Not provided | |
| | SVPB4 | Not provided | |
| | SVPB5 | U.S. | |
| | SVPB9 | Not provided | |
| | SVPB10 | Not provided | |
| | SVPB11 | Italy | |
| | SVPB12 | Italy | |
| | SVPB13 | U.S. | |
| | SVPB14 | Not provided | |
| | SVPB16 | Not provided | |
| | SVPB18 | Not provided | |
| | SVPB19 | Not provided | |
| C | DU123-6 | South Africa | Li et al. (2005) J. Virol. 80, 11776-11790. |
| | DU151-2 | South Africa | |
| | DU156-12 | South Africa | |
| | DU172-17 | South Africa | |
| | DU422 | South Africa | |
| | GXC44 | China | Dong et al (2003) J. Virol. 77, 3119-3130. |
| | 1-27 | China | Obtained from Subtype CRF_07 |
| | 2-138 | China | infected Chinese donors. |
| | 5-4 | China | |
| | 6-15 | China | |
| | 8-145 | China | |
| | 9-26 | China | |
| | 10-35 | China | |
| | 11-26 | China | |
| CRF01_AE | CM243 | Thailand | Dong et al. (2003) J. Virol. 77, 3119-3130. |
| | GXE14 | China | |
| D | NYU1545 | Cameroon | Cham et al. (2006) Virology 347, 36-51. |
| CRF11_cpx | CA1 | Cameroon | |
| H | VI525-5 | Africa | Beirnaert et al. (2000) J. Med. Virol. |
| CRF02_AG | 24/00/4 | Africa | 62, 14-24; Beirnaert et al. (2001) |
| F | 14/00/4 | Congo | Virology 281, 305-314. |
| CRF06_cpx | VI1793 | Africa | |

TABLE 2
Neutralization of Viruses Pseudotyped with Subtype
CRF07_B′C Envs by Sera from Donors from Xinjiang, China

| Env | 1/Serum Neutralizing Titer | | | | | | | | |
|---|---|---|---|---|---|---|---|---|---|
| Clone | 1 | 2 | 5 | 6 | 7 | 10 | 11 | 13 | 14 |
| 1-27 | 80 | <10 | 320 | 160 | <10 | 320 | 160 | 320 | 80 |
| 2-138 | <10 | <10 | 640 | 640 | <10 | 640 | 40 | 640 | 640 |
| 5-4 | <10 | <10 | <10 | <10 | <10 | 80 | <10 | <10 | <10 |
| 6-15 | <10 | <10 | 40 | <10 | <10 | <10 | <10 | <10 | <10 |
| 7-102 | <10 | <10 | <10 | <10 | 40 | 320 | 20 | <10 | <10 |
| 8-145 | <10 | <10 | 160 | 40 | <10 | 20 | <10 | 40 | 10 |
| 9-26 | <10 | <10 | 640 | 40 | <10 | 320 | 10 | 160 | 80 |
| 10-35 | <10 | <10 | <10 | <10 | <10 | 160 | <10 | 40 | <10 |
| 11-65 | <10 | <10 | 640 | 160 | <10 | 160 | <10 | 80 | 320 |

Homologous neutralization results are shown in bold. Titers are shown as 50% neutralization endpoints.

Neutralization Assays. Neutralization assays were conducted using pseudotyped viruses prepared by cotransfection of 293T cells with the plasmid pNL4-3.luc.E-R- and an env gene expressing plasmid. Assays were conducted in HOS cells using luminescence as an endpoint, as described previously (Cham et al, Virology on line 2006; Dong et al. (2003) J. Virol. 77, 3119-3130; Zhang et al. (1999) J. Virol. 73, 5225-5230; Quinnan et al. (1999) AIDS Res. Human Retrovir. 15, 561-570). The inventors have recently participated in a multicenter validation study comparing this assay to that described by Montefiori (Montefiori (2004) Evaluating neutralizing antibodies against HIV, SIV and SHIV in a luciferase reporter gene assay (Li et al. (2005) J. Virology 79, 10108)). These studies showed that the assays produce essentially identical results.

To determine neutralization, luminescence obtained in the presence of three control sera diluted 1:5 was averaged and compared to the mean for each individual serum. Test sera that inhibited ≧50%-75% were assigned titers of 1:5. Test sera that inhibited ≧75% were tested in serial dilutions in comparison to serial dilutions of concurrent control serum. This control serum was prepared by pooling serum from each of the control rabbits at the same sampling date.

Immunization Regimen. Adult New Zealand white rabbits were inoculated in triplicate at 0, 3, 6, and 28 weeks with volumes of Adjuvant A (which was prepared according to Example 5) with or without R2 envelope glycoprotein (30 μg gp120-R2 or 30 μg gp140-R2). The immunization and bleed schedule is shown in Table 3.

TABLE 3
Immunization and bleed schedule for phase 1 study

| Schedule day | Procedure |
|---|---|
| 0 | Pre-bleed and 1^{st} Immunization |
| 10 | Test bleed (1^{st} Bleed) |
| 21 | 2nd immunization |
| 31 | Test Bleed-I (2^{nd} Bleed) |
| 42 | 3rd immunization |
| 52 | Test Bleed-II (3^{rd} Bleed) |
| 197 | 4th immunization |
| 207 | Test Bleed-III (4^{th} Bleed) |

Immunizations. A 500 μl dose is administered as two intramuscular injections of 250 μl into each hind leg. For each of the first three immunizations, 500 μl of immunization mixture (500 μl per rabbit used), 300 μl of the concentrated Adjuvant A (1^{st} lot) was mixed with 200 μl of antigen (30 μg) in PBS. The fourth immunization mix was prepared using 250 μl of Adjuvant A and 250 μl PBS containing 30 μg of antigen. Subjects were immunized on days 0, 21, 42 and 197. Serum was collected on days 10, 31, 52 and 207. Sera were collected by bleed from the ear vein before the first vaccination and 10 days after each vaccination. Additionally, prebleeds of 10 ml of serum were obtained from all animals. Adjuvant concentrations were as follows: the 1^{st} lot of adjuvant was approximately 1.6× concentrated, the 2^{nd} lot of adjuvant was 2× concentrated. The rabbits in the gp140-immunized and control groups received two additional doses of immunogen, at 3 and 7 months after the fourth dose. Each of these doses consisted of the same materials as the previous doses, except that the last dose used the oil-emulsion adjuvant, AS03A (GlaxoSmithKline Biologicals, Rixensart, Belgium). Post sixth dose sera were used for IgG purification.

Enzyme Linked Immunosorbent Assays (ELISA). An antigen capture ELISA was used to determine serum Ig responses, as described previously (Dong et al. (2003) J. Virol. 77, 3119-3130; Quinnan et al. (2005) J. Virol. 79, 3358-3369).

Cloning of Envelope Genes. Viruses isolated from patients in Xinjiang Province, China were passaged once in PBMC from HIV-1 negative donors. Genomic DNA was extracted from the cells, and env gene cloning was accomplished using PCR, as previously described (see Zhang et al. (2002) J. Virol. 76, 644-655; Cham et al. (2005) Virology). Sequence encoding the HIV-2 strain 7312A gp160 was cloned using PCR from cell free virus stock supplied by the AIDS Research and Reference Reagent Program (Gao et al. (1994) J. Virol. 68, 7433-47), using methods described above for HIV-1.

Absorption of Rabbit Sera with 293T Cells and FAGS Analysis. Cells obtained by trypsinization from a confluent 75 cm² flask of 293T cells were resuspended in 400 μl of sera at final serum dilutions of 1:2.5. The suspensions were incubated at 4° C. for 3 hours with light rocking, cells were sedimented by centrifugation, and the absorption was repeated with new cells a second and third time. The third absorption was continued overnight. After each absorption, 5 μl of each serum was removed, diluted 1:200 and 1:1000 in PBS with 3% goat serum, and 100 μl of each was used to suspend 1.2×10⁵ 293T cells for FACS analysis. After 30 minutes on ice the cells were washed twice with PBS with 3% goat serum, and reacted with Biotin-SP-conjugated Anti Rabbit IgG (H+L) (Jackson ImmunoResearch), and then Streptavidin-PE (Sigma). The cells were washed and resuspended in 2% paraformaldehyde in PBS. Cells were analyzed on Beckman Coulter EPICS XL-MCL flow cytometer.

Purification of Serum IgG. Sera were clarified by centrifugation at 10,000 rpm for 15 minutes and then diluted 1:10 with PBS (pH 7.2). IgG was purified from diluted sera using the HiTrap protein G HP column (GE Healthcare Biosciences, Piscataway, N.J., USA), according to the manufacturer's instructions. Following purification, IgG was concentrated by centrifugation at 1500×g for 25 minutes using the centriprep centrifugal filter unit with Ultracel YM-30 membrane (Millipore, Billerica, Mass.). Concentration of purified IgG was determined using the NanoDrop® ND-1000 Spectrophotometer.

### Example 1

Neutralization of HIV-1 with Sera Obtained from Immunized Rabbits

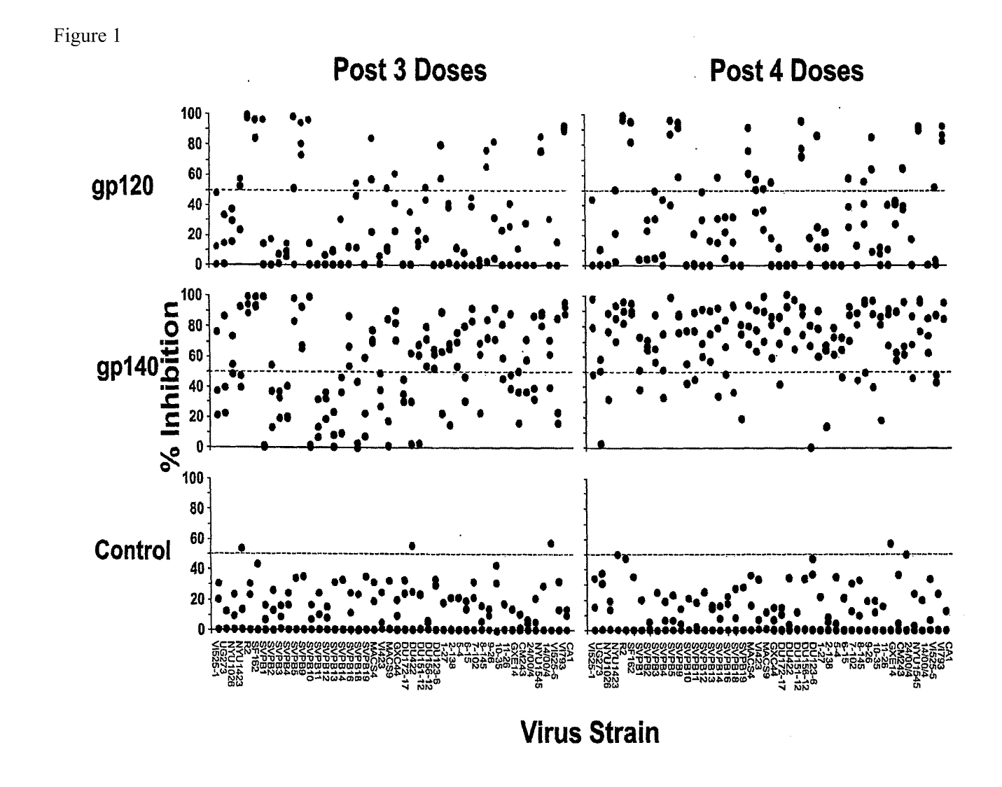

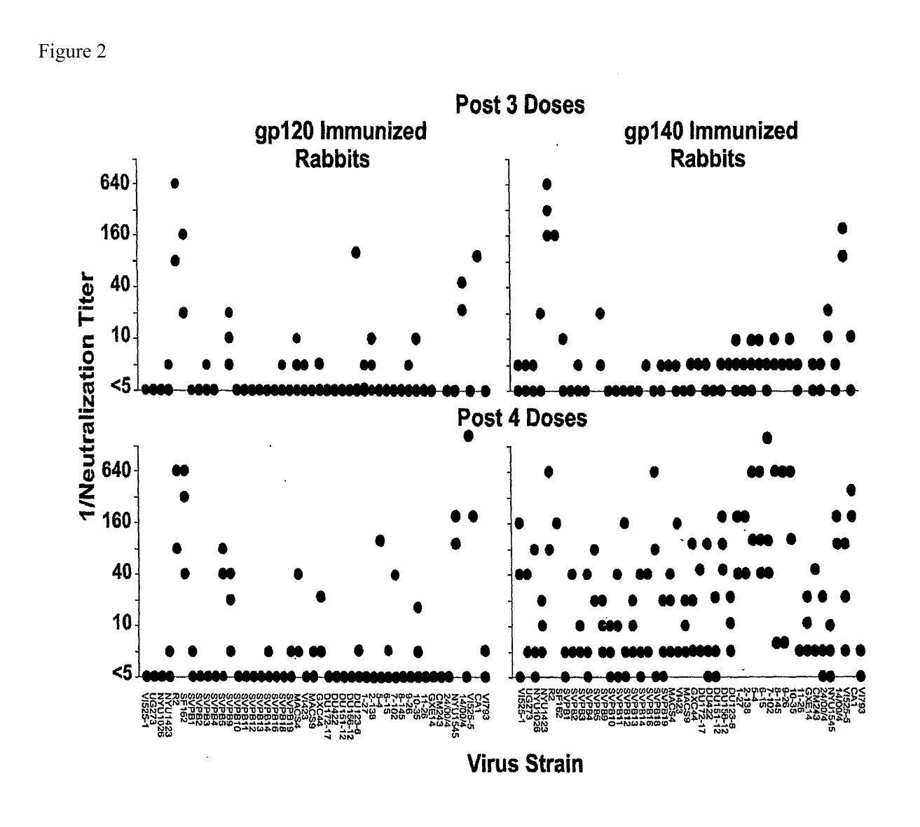

Results of neutralizing antibody assays performed on sera collected after the third and fourth doses of immunogen are shown in FIGS. 1 and 2. The results shown in FIG. 1 indicate the percentage (%) inhibition of luminescence in the presence of sera diluted 1:5 compared to virus infections conducted in the absence of serum. Neutralization results obtained using 46 different strains of HIV-1 are illustrated in FIG. 1. The calculated % inhibition by control sera exceeded 50% in only four of 176 possible combinations.

All of the strains of HIV-1 shown in FIG. 1 were neutralized >50% by sera from two or three of the rabbits after four doses of gp140, except for virus strain VI793 which was inhibited >50% by only one of three sera. Inhibition of infectivity was achieved less often by sera from rabbits immunized with gp120. After four doses >50% inhibition by two or three of the three sera was achieved only against the subtype B strains R2, SF162, SVPB9, MACS4, and MACS9, against the subtype C strains GXC44 and 10-35, against the subtype F strain 14/00/4, and the CRF11 strain CA1. It is notable that neutralization of these strains was observed after two or three doses of either gp120 or gp140, whereas neutralization of other strains was mostly evident only after four doses of immunogen.

The results shown in FIG. 2 indicate neutralization endpoint titers obtained after doses 3 and 4 of gp120 or gp140. Results were calculated as follows. Sera that inhibited luminescence more than 50% compared to the pool of three control sera at a 1:5 dilution were considered to have titers ≧1:5. If sera neutralized less than 80% at 1:5, they were considered to have titers of 1:5. Sera that neutralized greater than 80% at 1:5 were retested at serial dilutions beginning at 1:10 in parallel with pooled control sera. The mean luminescence results for each serum at each dilution were determined. The result obtained for each test serum was compared to the average result obtained for the pooled control sera at the same dilutions. Test sera that inhibited luminescence ≧50% (upper panels) or ≧80% (lower panel) compared to the average for comparable dilutions of pooled control sera were considered neutralizing at that dilution. The last dilution considered to be neutralizing was assigned as the endpoint. The variation among the results for control sera at the 1:5 dilution was sufficiently limited that inhibition of luminescence by ≧50% of the control average by individual control sera was observed in only four of 276 possible events. In contrast, after four immunizations, the sera from either two or all three of the gp120 immunized rabbits inhibited ≧50% in the case of nine strains (strains R2, SF162, SVPB5, SVPB9, MACS4, GXC44, 10-35, 14/00/4, and CA1). The frequency of neutralization was significantly greater by Chi Square test by sera from gp120 immunized than control rabbits after both the third (p=1.9×10⁻⁶) and fourth (p=1.7×10⁻⁸) doses. Immunization with gp140 resulted in more broadly cross-reactive neutralization than immunization with gp120. After three doses, either two or three of the sera from the gp140-immunized rabbits neutralized 23 strains of HIV-1, and after four doses, all but one of the strains was neutralized by at least two of the sera. The differences after three (p=2.98×10⁻⁶) and four (p=4.1×10⁻²⁴) doses were statistically significant.

The patterns observed when neutralization endpoint titers were compared among the different strains were similar to those observed in comparison of % inhibition at a 1:5 dilution. The results shown in FIG. 2 demonstrate that one or more sera from animals receiving gp140 neutralized each virus. Titers tended to increase after dose 4 compared to after dose 3. Notably, 43 of the HIV-1 strains were neutralized at titers ≧1:10, and 39 at titers ≧1:20 by at least one of the sera from gp140 immunized rabbits (particularly, rabbit 4). Titers tended to increase after the fourth dose compared to the third, and to be greater after immunization with gp140 than gp120. Strains that were neutralized by two or three of the sera from gp120-immunized rabbits were neutralized at similar titers by sera from rabbits immunized with gp120 and gp140. Virus strains that were neutralized by sera from rabbits immunized with gp120 tended to be neutralized more often after two or three immunizations and at higher titers than strains not neutralized by those sera.

### Example 2

### Neutralization of Wild Type and Mutant Strains of R2 and 14/00/4

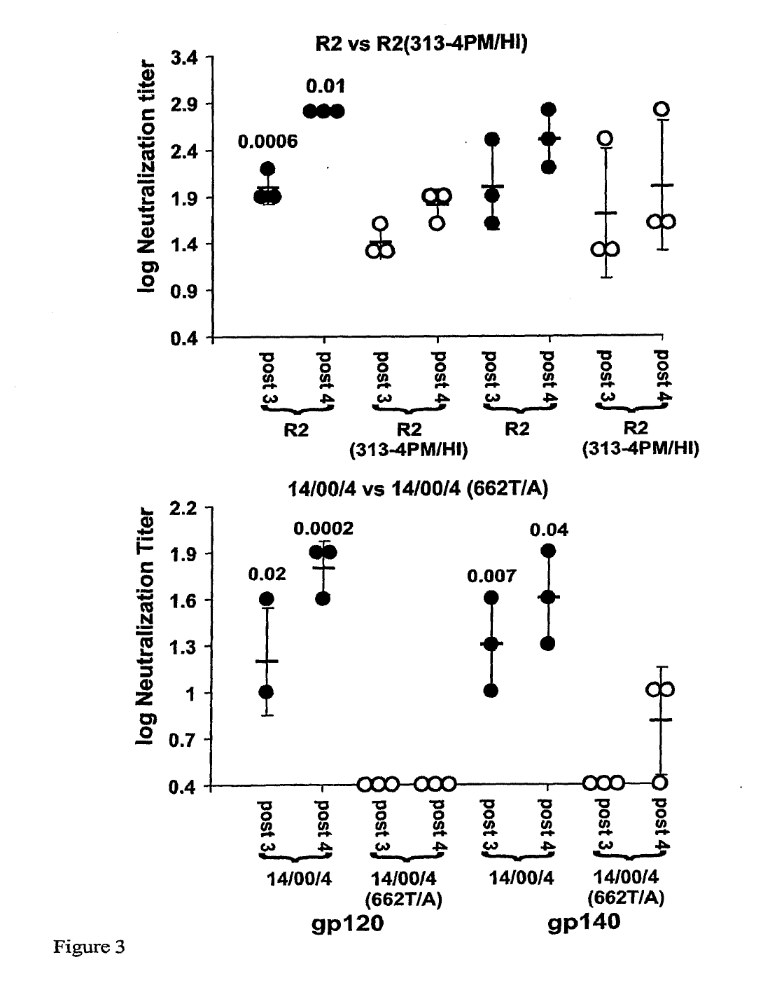

Two of the strains tested for neutralization originate from donors with broadly cross-reactive neutralizing antibodies and they have very unusual amino acid sequences that may be related to the breadth of cross-reactivity of neutralizing antibodies in the donors from which they came. One of these strains is R2, the strain used for immunization. The R2 envelope glycoprotein mediates CD4-independent infection, a property that depends on the proline-methionine sequence at residues 313-4 of its V3 loop. The 14/00/4 envelope glycoprotein is resistant to neutralization by monoclonal antibodies directed against many gp120 epitopes, but is highly sensitive to neutralization by monoclonal antibodies directed against membrane proximal epitopes, 2F5 and 4E10. This sensitivity depends upon a very rare tyrosine residue at position 662. Viruses pseudotyped with each of these glycoproteins were highly sensitive to neutralization by sera from both gp120 and gp140 immunized rabbits (FIG. 1). Each of these prototype strains and corresponding mutants were compared for neutralization by the rabbit sera, as shown in FIG. 3. Mutation of residues 313-4 of the R2 envelope glycoprotein caused it to become significantly more resistant to neutralization by sera from rabbits immunized with gp120, and somewhat more resistant to sera from rabbits immunized with gp140, but not significantly so. The difference in sensitivity of the wild type and R2 (313-4/PM) variants to neutralization by the sera from rabbits immunized with gp120 was approximately 6- and 25-fold after three and four doses, respectively. The difference in neutralization of these two strains by gp140 immune sera was about 2- and 3.2-fold, after three and four doses, respectively. The 14/00/4 (662T/A) mutant was significantly more resistant to neutralization by both gp120 and gp140 immune sera than was the wild type 14/00/4 strain. The gp120-immune sera neutralized wild-type 14/00/4 approximately 6.4 and 25-fold more than the mutant after three and four doses, respectively, while the gp140-immune sera neutralized the wild-type approximately 8 and 6.4-fold more. Both mutant variants were neutralized by the post fourth dose, gp140 immune sera.

### Example 3

Sera from gp140 or gp120 Immunized Rabbits Neutralize Viruses Pseudotyped with Envelope Glycoproteins from Pathogenic SHIV and HIV Strains

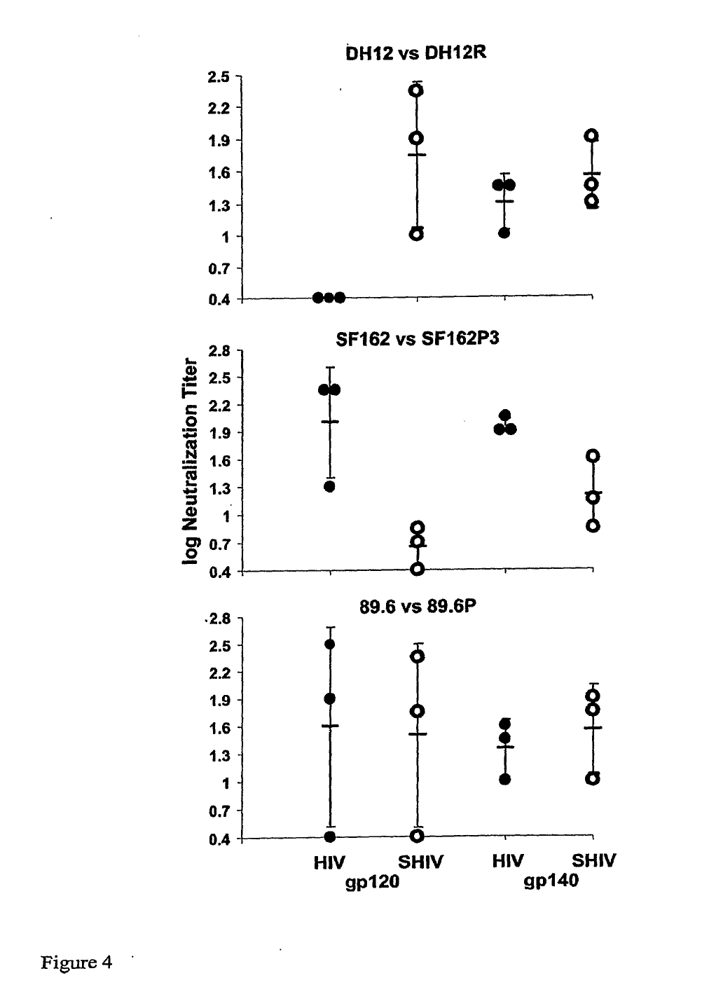

Comparative neutralization of viruses pseudotyped with envelope glycoproteins from pathogenic SHIV and the HIV strains DH12, SF162, and 89.6 from which they were derived is shown in FIG. 4. The results shown are averages of results obtained from two independent experiments, each done in triplicate. The two experiments produced similar results. All three strains of SHIV and HIV were neutralized by all three sera from gp140-immunized rabbits. One of the three strains, SF162P3 was about four-fold more resistant to neutralization by these sera than the corresponding HIV-1 strain. The other two SHIV were neutralized comparably to the corresponding HIV-1 strains by the sera from gp140-immunized rabbits. Each HIV-1/SHIV pair differed in comparative neutralization by the gp120 immune sera. Those sera neutralized both the HIV-1 and SHIV variants of strain 89.6, only the HIV-1 variant of strain SF162, and only the SHIV variant of strain DH12.

### Example 4

Sera from gp140 Immunized Rabbits Bind HIV-1 Strains R2, 14/00/4, and CM243

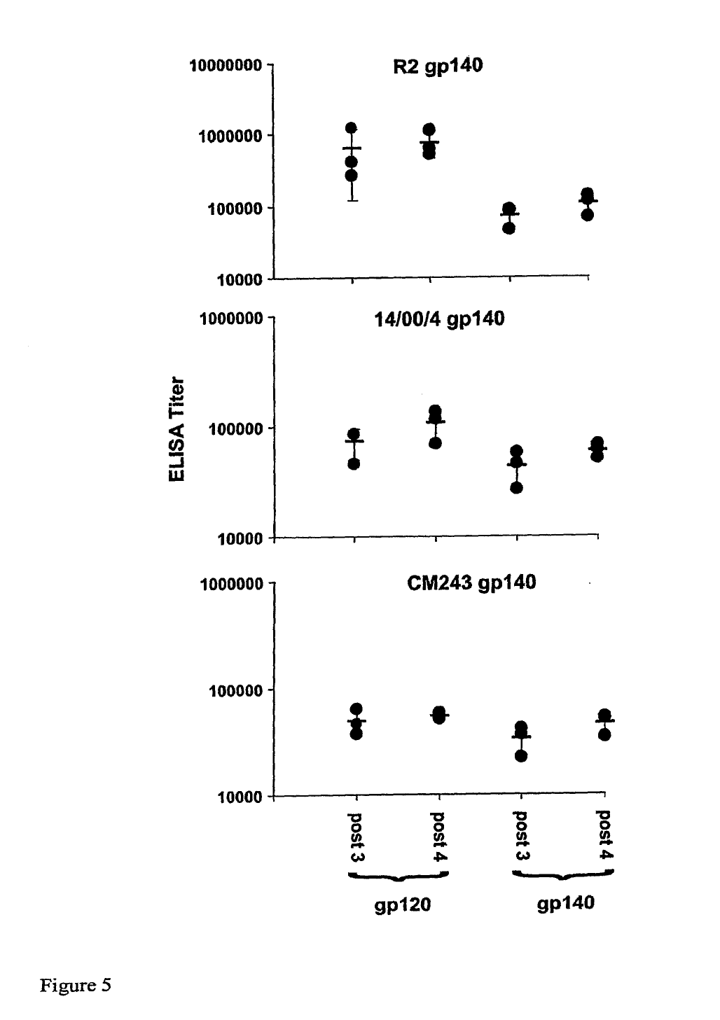

Results of antibody testing by ELISA are shown in FIG. 5. Sera obtained after the third and fourth doses of immunogen were tested for antibodies to gp140 of strains R2, 14/00/4, and CM243. The procedures used have been described elsewhere (Quinnan et al. (2005) J. Virol. 79, 3358-3369). The rabbits immunized with gp120 developed higher R2gp140 binding titers than those immunized with gp140. There was no significant increase in R2gp140 binding antibodies after the fourth dose of gp120, but there was a significant increase after the fourth dose of gp140 (p<0.05, student t test). The rank order of binding antibody titers against the different envelopes was R2>14/00/4>CM243. The trend for greater binding antibody titers after gp120 immunization was evident for 14/00/4 glycoprotein, but not CM243. Small, but significant increases in 14/00/4 binding antibodies were noted after the fourth dose of gp120 (p=0.03), and in CM243 binding antibodies were noted after the fourth dose of gp140 (p=0.003).

### Example 5

### Preparation of Oil in Water Emulsion

The preparation of oil in water emulsion followed the protocol as set forth in WO 95/17210. The emulsion contains 42.72 mg/ml Squalene, 47.44 mg/ml tocopherol, and 19.4 mg/nil Tween 80. The resulting oil droplets have a size of approximately 180 nm. Tween 80 was dissolved in phosphate buffered saline (PBS) to give a 2% solution in the PBS. To provide a 100 ml two-fold concentrate emulsion, 5 g of DL alpha tocopherol and 5 ml of squalene were first vortexed until mixed thoroughly. 90 ml of PBS/Tween solution was then added and mixed thoroughly. The resulting emulsion was then passed through a syringe and finally microfluidised by using an M110S microfluidics machine. The resulting oil droplets have a size of approximately 180 nm.

Preparation of Oil in Water Emulsion with QS21 and MPL (Adjuvant A)

Sterile bulk emulsion was added to PBS to reach a final concentration of 500 μl of emulsion per ml (v/v). 3 D-MPL was then added to reach a final concentration of 100 μg per nil. QS21 was then added to reach a final concentration of 100 μg per ml. Between each addition of component, the intermediate product was stirred for 5 minutes. Fifteen minutes later, the pH was checked and adjusted if necessary to 6.8+/−0.1 with NaOH or HCl. This mixture is referred to as adjuvant A.

### Example 6

### Preparation of Liposomal MPL

A mixture of lipid (such as phosphatidylcholine either from egg-yolk or synthetic) and cholesterol and 3 D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 10 mg/ml. The final concentration of MPL is 2 mg/ml.

The liposomes have a size of approximately 100 nm and are referred to as SUV (for small unilamelar vesicles). The liposomes by themselves are stable over time and have no fusogenic capacity.

### Preparation of Adjuvant B

Sterile bulk of SUV was added to PBS to reach a final concentration of 100 μg/ml of 3D-MPL. PBS composition was Na₂HPO₄: 9 mM; KH₂PO₄: 48 mM; NaCl: 100 mM and pH 6.1. QS21 in aqueous solution was added to the SUV to reach a final concentration of 100 μg/ml of QS21. This mixture is referred to as Adjuvant B. Between each addition of component, the intermediate product was stirred for 5 minutes. The pH was checked and adjusted if necessary to 6.1+/−0.1 with NaOH or HCl.

### Example 7

Sera from gp140 and gp120 Plus Adjuvant B Immunized Rabbits Produce Antibodies Capable of Neutralizing HIV-1 Primary Isolates.

Rabbits were immunized as shown in Table 3. Adjuvant B was prepared as set out in Example 6.

TABLE 4
Groups of rabbits immunized with the various adjuvanted R2 proteins

| | Number | | Production | |
|---|---|---|---|---|
| Group | of animals | Antigen | system | Adjuvant |
| 1 | 2 | 20 μg R2 gp140 | vaccinia | Adjuvant B |
| 2 | 3 | 20 μg R2 gp120 | CHO | Adjuvant B |
| 3 | 3 | 20 μg R2gp140ΔCS | CHO | Adjuvant B |

Immunizations were performed on days 0, 21 and 42, and serum samples were taken on day 56 (14dpIII). These sera were sent to Monogram Biosciences (San Francisco, USA) to test for the presence and titers of neutralizing antibody activity to a series of Clade B and C HIV-1 primary isolates.

As shown in Table 5 the CHO produced R2 gp120 specific serum is able to neutralize 3 out of the 11 clade B viruses, and none of the clade C viruses. This is similarly observed for the CHO produced R2 gp140 specific serum. The vaccinia produced R2gp140 specific serum is able to neutralize 3 out of the 11 clade B, and also one of the 6 clade C viruses.

The data in Table 5 are presented as the titer where 50% neutralization is observed for that specific virus. Positivity (shown as bold and underlined data) is defined as being above the Pre+3sd cutoff for that particular virus.

TABLE 5
N50 neutralization data for 14dpIII serum produced in rabbits immunized
with R2 gp140 plus adjuvant B or R2 gp120 plus adjuvant B.

| | | CHO R2 gp140 | | | CHO R2 gp120 | | | Vaccinia R2 gp140 | |
|---|---|---|---|---|---|---|---|---|---|
| | | Adjuvant B | | | Adjuvant B | | | Adjuvant B | |
| | Virus | TA733 | TA7354 | TA735 | TA730 | TA731 | TA732 | TA706 | TA707 |
| Clade | 692 | 19 | 28 | 30 | 35 | 24 | 38 | 15 | 16 |
| B | 1196 | 30 | 43 | 51 | 74 | 49 | 75 | 31 | 41 |
| | 92HT594 | 16 | 21 | 25 | 30 | 18 | 37 | <10 | <10 |
| | 93US073 | 13 | 16 | 15 | 23 | 12 | 22 | <10 | <10 |
| | Bal | 21 | 43 | 17 | 60 | 29 | 58 | 24 | 26 |
| | BX08 | 27 | 36 | 24 | 42 | 25 | 41 | 19 | 17 |
| | JRCSF | <10 | <10 | <10 | 14 | <10 | 16 | <10 | <10 |
| | NL43 | 269 | 538 | 180 | 626 | 802 | 1302 | 907 | 925 |
| | QZ4589 | 40 | 61 | 42 | 7 | 44 | 102 | 39 | 64 |
| | SF162 | 504 | 1071 | 485 | 1982 | 740 | 1798 | 1160 | 1428 |
| | W61D | 92 | 243 | 127 | 73 | 124 | 1087 | 648 | 390 |
| Clade | 301960 | 14 | 27 | 19 | 43 | 29 | 44 | 12 | 11 |
| C | 98CN006 | 21 | 29 | 44 | 46 | 37 | 60 | 28 | 31 |
| | 93IN101 | 15 | 20 | 21 | 29 | 20 | 38 | <10 | <10 |
| | 97ZA009 | <10 | 11 | <10 | 19 | <10 | 21 | <10 | <10 |
| | 98TZ013 | 16 | 25 | 34 | 43 | 33 | 58 | <10 | <10 |
| | 98TZ017 | 20 | 32 | 52 | 54 | 36 | 65 | 19 | 29 |
| | AMLV | 10 | 11 | 16 | 37 | 19 | 43 | <10 | <10 |

### Example 8

Sera from HIV-1 Strain R2 gp140 and gp120 Plus Adjuvant B Immunized Guinea Pigs Produces Antibodies Capable of Neutralizing HIV-1 Primary Isolates.

Guinea pigs were immunized as shown in Table 6. Immunizations were performed on days 0, 21 and 42, and serum samples were taken on day 56 (14dpIII). These sera were sent to Monogram Biosciences (San Francisco, USA) to test for the presence and titers of neutralizing antibody activity to a series of clade B and C HIV-1 primary isolates.

TABLE 6
Groups of guinea pigs immunized with the various adjuvanted R2 proteins

| | Number | | Production | |
|---|---|---|---|---|
| Group | of animals | Antigen | system | Adjuvant |
| 1 | 2 | 4 μg R2 gp120 | vaccinia | Adjuvant B |
| 2 | 2 | 4 μg R2 gp140 | vaccinia | Adjuvant B |
| 3 | 3 | 4 μg R2 gp120 | CHO | Adjuvant B |
| 4 | 3 | 4 μg R2 gp140ΔCS | CHO | Adjuvant B |

As shown in Table 7, the CHO produced R2 gp120 specific serum is able to neutralize 2 and 4 out of the 11 clade B and none of the clade C. The CHO produced R2 gp140 specific serum is able to neutralize between 6 and 8 out of the 11 clade B viruses, and none of the clade C viruses.

The vaccinia produced R2 gp140 specific serum is able to neutralize 3 out of the 11 clade B, and none of the 6 clade C viruses. While the vaccinia produced R2 gp120 specific serum is able to neutralize 7 out of the 11 clade B viruses, and with one out of the two guinea pigs 2 of the 6 clade C viruses.

The data in Table 7 are presented as the titer where 50% neutralization is observed for that specific virus. Positivity (shown as bold and underlined data) is defined as being above the Pre+3sd cutoff for that particular virus.

TABLE 7
N50 neutralization data for 14dpIII serum produced in guinea pigs immunized
with R2 gp140 plus adjuvant B or R2 gp120 plus adjuvant B.

| | | CHO R2 gp140 | | | CHO R2 gp120 | | | Vaccinia R2 gp140 | | Vaccinia R2 gp120 | |
|---|---|---|---|---|---|---|---|---|---|---|---|
| | | Adjuvant B | | | Adjuvant B | | | Adjuvant B | | Adjuvant B | |
| | Virus | A | B | C | D | E | F | G | H | I | K |
| Clade | 692 | <10 | <10 | <10 | <10 | <10 | <10 | 14 | <10 | <10 | 10 |
| B | 1196 | 106 | 47 | 151 | <10 | 19 | <10 | 133 | 70 | 70 | 90 |
| | 92HT594 | 13 | 12 | <10 | <10 | 15 | <10 | 31 | 13 | 21 | 17 |
| | 93US073 | <10 | <10 | <10 | <10 | <10 | <10 | 12 | <10 | <10 | <10 |
| | Bal | 83 | 25 | 87 | <10 | 10 | <10 | 63 | 42 | 36 | 121 |
| | BX08 | 50 | 12 | 47 | <10 | <10 | <10 | 27 | 16 | 36 | 76 |
| | JRCSF | <10 | <10 | <10 | <10 | <10 | <10 | <10 | <10 | <10 | <10 |
| | NL43 | 1600 | 1492 | 6175 | 5349 | 72 | 136 | 1134 | 2616 | 2704 | 14181 |
| | QZ4589 | 85 | 34 | 181 | 21 | 18 | <10 | 95 | 108 | 160 | 61 |
| | SF162 | 3801 | 1824 | 5252 | 615 | 327 | 133 | 6809 | 3516 | 7656 | 45048 |
| | W61D | 1232 | 829 | 2818 | 519 | 139 | 138 | 1958 | 2137 | 1246 | 61 |
| Clade | 301960 | 11 | <10 | <10 | <10 | <10 | <10 | 78 | 141 | 24 | 17 |
| C | 98CN006 | <10 | <10 | <10 | <10 | <10 | <10 | 35 | 31 | 12 | 11 |
| | 93IN101 | <10 | <10 | <10 | <10 | <10 | <10 | 29 | 17 | <10 | <10 |
| | 97ZA009 | <10 | <10 | <10 | <10 | <10 | <10 | 51 | 19 | <10 | <10 |
| | 98TZ013 | <10 | <10 | <10 | <10 | <10 | <10 | 36 | 34 | <10 | <10 |
| | 98TZ017 | <10 | <10 | <10 | <10 | <10 | <10 | 36 | 40 | 22 | 11 |
| | AMLV | <10 | <10 | <10 | <10 | <10 | <10 | 62 | 240 | <10 | <10 |

A = 50428021
B = 50428022
C = 50428023
D = 50428011
E = 50428012
F = 50428013
G = 50318091
H = 50318092
I = 50318101
K = 50318102

The data from Examples 7 and 8 suggest that the R2 proteins formulated with adjuvant B are able to induce the production of antibodies capable of neutralising HIV-1 primary isolates.

### Example 9

Differential Appearance of Antibodies that Neutralize Viruses Sensitive and Resistant to gp120-Induced Antibodies.

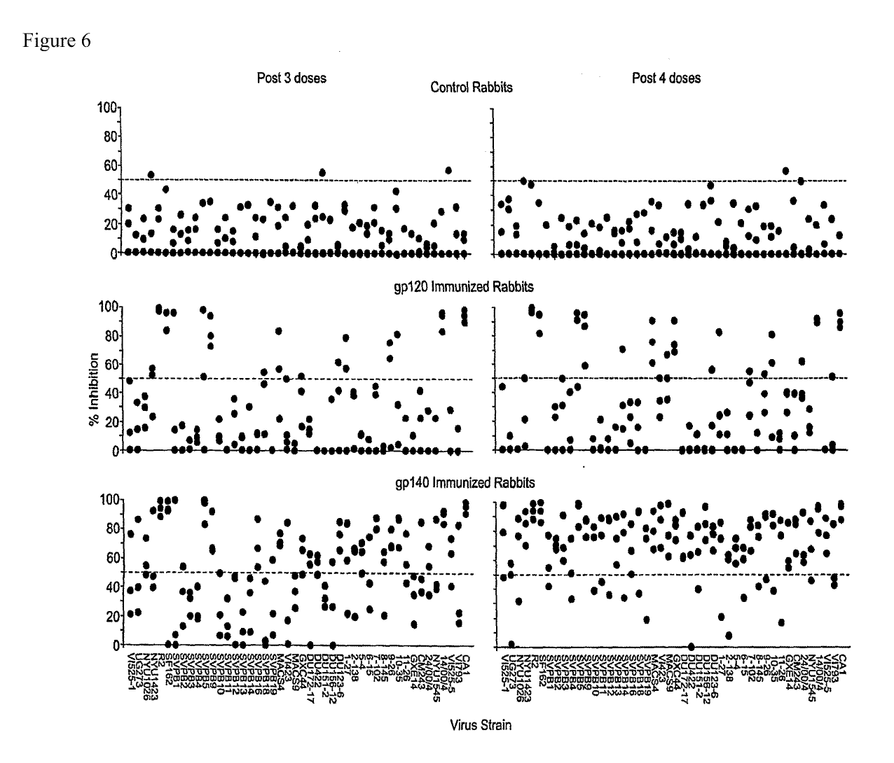

Antibodies that neutralized the nine strains that were sensitive to gp120-induced antibodies developed more rapidly than antibodies that neutralized strains that were only sensitive to gp140-induced antibodies, as further discussed below and shown in FIG. 6. The frequency with which viruses were neutralized by sera from gp120 immunized rabbits was similar after three or four immunizations, while the frequency increased substantially after four, compared to three doses of gp140 (X², p=4.3×10⁻⁹). The gp120 induced neutralizing responses actually approached maximal levels after the second dose of immunogen, just 4.5 weeks following the start of the immunization protocol.

### Example 10

### HIV-1 Specificity of Neutralizing Antibody Responses.

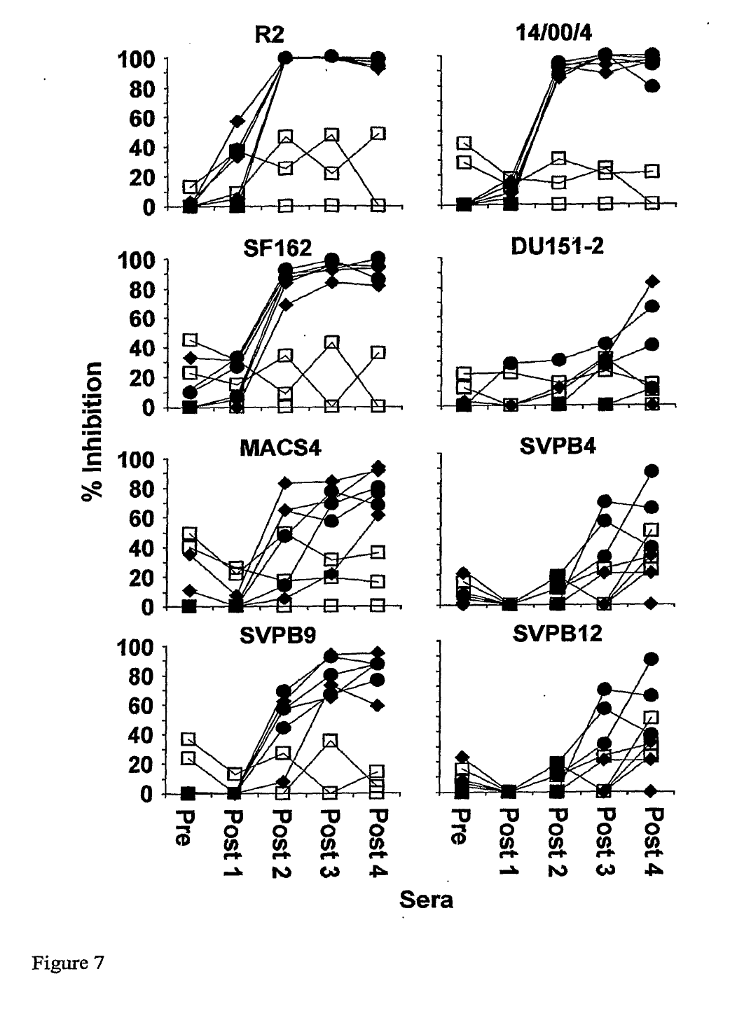

Sera were tested for neutralization of viruses pseudotyped with HIV-2 Env and VSV G protein, both produced by transfection of 293T cells, as shown in FIG. 7A. Compared to control sera, the post fourth dose sera from the immunized rabbits did not neutralize either HIV-2 or VSV. Similar results were observed in repeat experiments. In experiments not shown, virus pseudotyped with Nipah virus F and G proteins was prepared and tested for neutralization by the same sera. No significant differences were observed.

The possibility that the virus inhibitory activity in the rabbit sera was due to antibodies directed against cell antigens was investigated. In preliminary experiments using fluorescence activated cell sorting (FACS) significant binding activity against both BSC-1 and 293T cells was found in the sera from the gp120 and gp140 immune rabbits, although the levels were greater in the gp140-immune sera. The level of cell binding IgG in sera from rabbits immunized with regimens that induced less cross-reactive neutralizing activity was investigated. The levels in the gp140_{R2}-immune sera were similar to those in sera from rabbits immunized with HIV-1 gp140_{CM243} in RiBi adjuvant, which did not induce neutralizing antibodies (data not shown). They were also similar to those in sera from rabbits immunized with a regimen that involved priming with Venezuelan Equine Encephalitis virus replicon particles expressing gp160_{R2} followed by boosting with gp140_{R2} in RiBi adjuvant (Dong et al. (2003) J. Virol. 77, 3119-3130). Sera from these latter rabbits have antibodies that neutralize several strains of HIV-1, but not a number of neutralization resistant strains shown in FIG. 6 (Dong et al. (2003) J. Virol. 77, 3119-3130). These results demonstrated that the presence of 293T cell-binding immunoglobulin in sera did not correlate with the cross reactivity of the neutralizing response to gp140.

In view of those preliminary FACS data, the experiment shown in FIGS. 7B and 7C was conducted. Sera from after four doses of gp140_{R2} and pooled sera collected before immunization from the same rabbits were absorbed with 293T cells and tested for cell binding activity in FACS and for neutralizing activity. Absorptions were conducted at high serum concentrations, so that sera could be used subsequently in neutralization assays. At such high serum concentrations exhaustive removal of cell binding activity could not be accomplished. However, substantial reduction in cell binding activity was achieved by three sequential absorptions, since there was almost no activity remaining when sera was diluted 1:1000 for testing in FACS assay, and significant reduction was reflected in assays that were conducted using 1:200 dilutions of the rabbit sera (FIG. 7B). Interestingly, pre-immunization sera also possessed significant cell binding activity, detected in sera diluted 1:200, which was removed by absorption. Neutralization assay was conducted using the thrice-absorbed serum from Rabbit 4, shown in FIG. 7C. The absorption procedure caused no significant reduction of neutralizing activity against either the subtype B or C virus tested, strains SVPB11 and DU123, respectively, both of which were resistant to neutralization by antibodies induced by gp120_{R2}.

### Example 11

### Neutralization of Primary Viruses is Mediated by Immunoglobulin G (IgG).

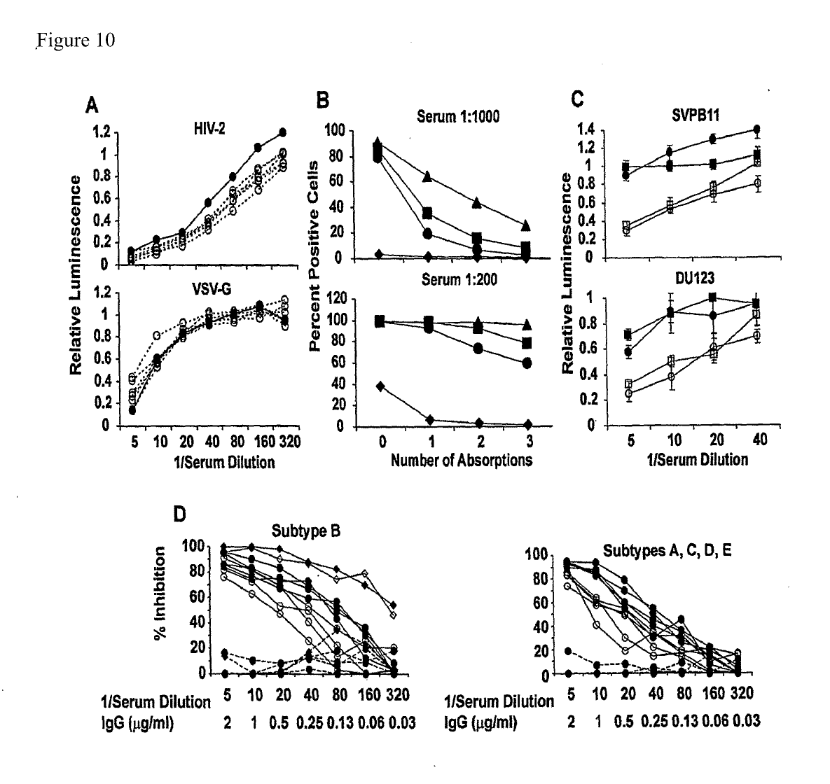

Insufficient serum volumes were available from the post fourth dose bleeds to permit purification and neutralization testing of IgG fractions. Therefore, sera collected after two more immunizations, as previously described, were used for this purpose. Sera and IgG fractions were tested in parallel for neutralization of the viruses shown in FIG. 7D. The IgG concentrations were adjusted to be approximately equivalent to the concentration of IgG in rabbit serum (i.e., 10 μg/ml of undiluted serum). The neutralizing activity of the serum and IgG were identical against the R2 strain, while the IgG was equivalent or superior to serum against five additional subtype B strains, two subtype C strains and single strains of subtypes C, D, and E. All of the strains shown in FIG. 10A, except R2, were resistant to neutralization by gp120-induced antibodies. No neutralizing activity was present in the IgG from the control rabbits.

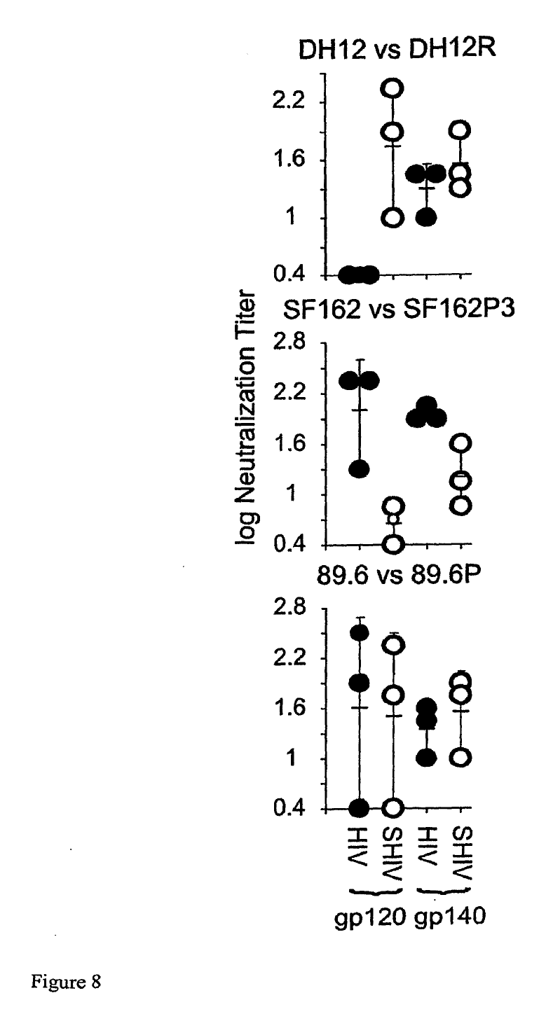

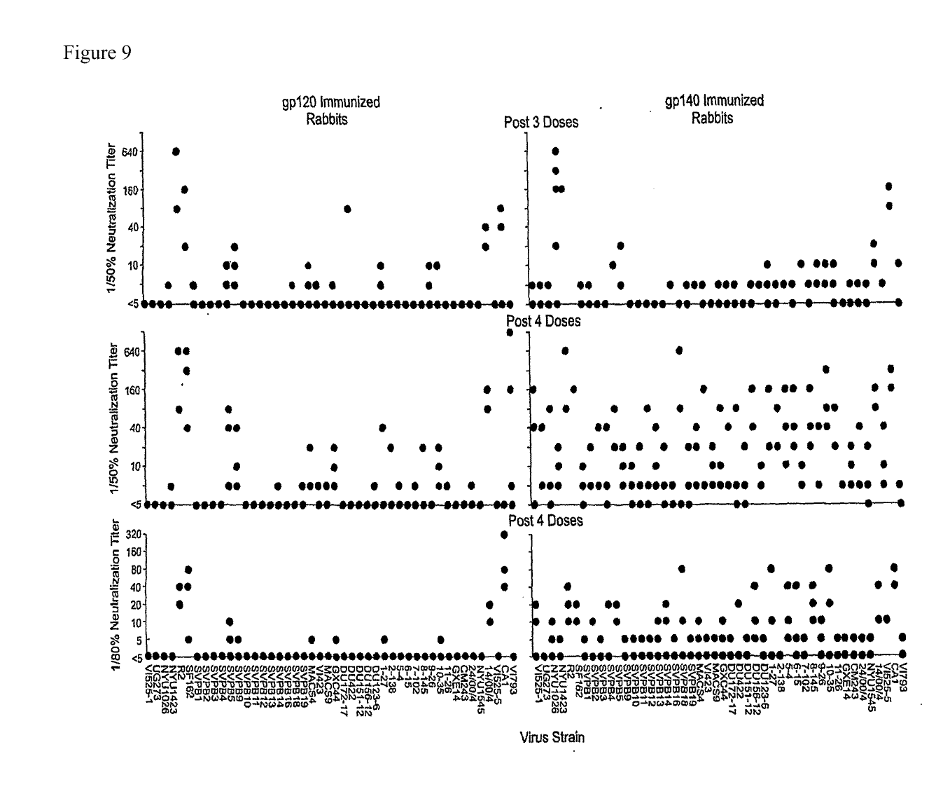

HIV-1 specificity of the neutralizing activity in the post sixth dose serum was evaluated, as described in below and shown in FIGS. 8 and 9. Both the serum and IgG from Rabbit 4 contained 293T cell binding activity and VSV neutralizing activity. Serial absorption with 293T cells removed most of the cell binding activity and all of the VSV neutralizing activity from the IgG, and significantly reduced both in the serum. Absorption did not affect neutralization of the HIV-1 strains tested. Thus, the evidence indicated that the IgG contained antibodies that specifically neutralized HIV-1 strains that were generally neutralization resistant strains.

HIV-1 Specific IgG Neutralizing Activity in Post Sixth Dose Rabbit Serum. The reactivity of IgG in post sixth dose rabbit serum with cells, VSV, and HIV-1 was tested to evaluate the specificity of the IgG mediated neutralization of HIV-1. The sera and IgG from Rabbit 4 had significant cell binding activity, while little was detected in the control sera, as shown in FIG. 8. Successive absorptions with 293T cells resulted in progressive, significant reduction in binding activity in both, with almost complete removal of binding activity in the IgG fraction. The absorbed and unabsorbed sera and IgG were tested for neutralization of VSV, SVPB19 (Subtype B), and DU422 (Subtype C), as shown in FIG. 9. Unabsorbed sera and IgG both inhibited infectivity of VSV, but the inhibitory effect was completely removed from the IgG and reduced in the serum by absorption to 293T cells. In contrast, absorption had no effect on HIV-1 neutralizing activity of either the serum or IgG. The results demonstrate that the six-dose immunization regimen did induce IgG responses against antigens on the surface of 293T cells, and that removal of those antibodies by absorption to 293T cells eliminated binding to 293T cells as well as neutralization of VSV. However, since the removal of the cell binding and VSV neutralizing activity had no effect on IgG neutralization of HIV-1, the data support the interpretation that the immunization regimen induced HIV-1 specific IgG with broadly cross neutralizing activity:

As evident from the examples, the gp140_{R2} immunogen induced antibodies that achieved 50 percent neutralization of 48/48, and 80 percent neutralization of 43/46 primary strains of diverse HIV-1 subtypes tested. The strains tested included members of standard panels of subtype B and C strains, and other diverse strains known to be neutralization resistant. The gp120_{R2} induced antibodies that neutralized 9/48 of the same strains. Neutralization was IgG mediated and HIV-1 specific.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims.

1. An immunogenic composition comprising an isolated HIV envelope protein capable of inducing the production of a cross-reactive neutralising anti-serum against multiple strains of HIV-1 in vitro wherein the V3 region of the HIV envelope protein comprises amino acids 313 to 325 of SEQ ID NO: 1 or immunogenic fragments thereof; and an adjuvant comprising a Toll like receptor (TLR) 4 ligand, in combination with a saponin.

2. An immunogenic composition comprising an isolated HIV envelope protein capable of inducing the production of a cross-reactive neutralising anti-serum against multiple strains of HIV-1 in vitro wherein HIV envelope protein comprises an amino acid sequence with at least 92% identity to SEQ ID NO: 1; and an adjuvant comprising a Toll-like receptor (TLR) 4 ligand, in combination with a saponin.

3. An immunogenic composition according to claim 1 wherein the Toll like receptor (TLR) 4 ligand is a lipid A derivative.

4. An immunogenic composition according to claim 3 wherein the lipid A derivative is monophosphoryl lipid A.

5. An immunogenic composition according to claim 4 wherein the monophosphoryl lipid A is 3 Deacylated monophosphoryl lipid A (3 D-MPL).

6. An immunogenic composition according to claim 3 wherein the lipid A derivative is selected from the group consisting of OM174, OM 294 DP, and OM 197 MP-Ac DP.

7. An immunogenic composition according to claim 1 wherein the Toll like receptor (TLR) 4 ligand is an alkyl glucosaminide phosphate.

8. An immunogenic composition according to claim 1 wherein the saponin is QS-21 or QS-7.

9. An immunogenic composition according to claim 8 wherein the saponin is presented in the form of a liposome, ISCOM or an oil in water emulsion.

10. An immunogenic composition according to claim 2 wherein the HIV envelope protein comprises an amino acid sequence has at least ninety five percent identity to SEQ ID NO: 1.

11. An immunogenic composition according to claims 2 wherein the HIV envelope protein comprises the amino acid sequence of SEQ ID NO: 1.

12. An immunogenic composition according to claim 1 wherein the adjuvant comprises QS21, MPL and tocopherol in an oil in water emulsion.

13. An immunogenic composition according to claim 1 wherein the adjuvant comprises liposomal QS21 and MPL wherein the liposomes have a size of approximately 100 nm.

14. An immunogenic composition according to claim 1 further comprising aluminium hydroxide or aluminium phosphate.

15. A method of inducing an immune response by administration of an immunogenic composition according to claim 1 to a human in need thereof.

16. (canceled)
